(19) United States
(12) Patent Application Publication
(10) Pub. No.: US 2026/0216305 A1
(43) Pub. Date: Jul. 30, 2026

(54) TAU PEPTIDE IMMUNOGEN CONSTRUCTS

(71) Applicant: Vaxxinity, Inc., Merritt Island, FL (US)

(72) Inventors: Justin Boyd, Merritt Island, FL (US); Jean-Cosme Dodart, Merritt Island, FL (US)

(21) Appl. No.: 18/993,912

(22) PCT Filed: Jul. 14, 2023
(86) PCT No.: PCT/US23/27832

§ 371 Date: Jan. 13, 2025

Related U.S. Application Data
(60) Provisional application No. 63/389,241, filed on Jul. 14, 2022.
(60) Provisional application No. 63/393,533, filed on Jul. 29, 2022.

(51) Int. Cl. A61K 39/00; A61K 38/00; A61P 25/28; C07K 14/47

(52) U.S. Cl. CPC A61K 39/0007; A61P 25/28; C07K 14/4711; A61K 38/00; A61K 2039/55505; A61K 2039/55561; A61K 2039/575; C07K 2319/40

(57) ABSTRACT

The present disclosure is directed to individual peptide immunogen constructs targeting portions of the Tau protein for the treatment and/or prevention of tauopathies. The present disclosure is also directed to compositions containing the peptide immunogen constructs, methods of making and using the peptide immunogen constructs, and antibodies produced by the peptide immunogen constructs.

### CROSS-REFERENCE TO RELATED APPLICATIONS

This application is a § 371 U.S. national stage application of International Patent Application No. PCT/US23/27832, filed Jul. 14, 2023, which claims the benefit of U.S. Provisional Patent Application No. 63/389,241, filed Jul. 14, 2022 and U.S. Provisional Patent Application No. 63/393,533, filed Jul. 29, 2022, the entire contents of each of which are incorporated by reference herein in their entireties.

### SEQUENCE LISTING

The instant application contains a Sequence Listing which has been submitted electronically in XML format and is hereby incorporated by reference in its entirety. Said XML copy, created on Mar. 1, 2026, is named AXε-10701.xml and is 170,908 bytes in size.

### FIELD OF THE INVENTION

This disclosure relates to peptide immunogen constructs based on portions of the Tau protein and formulations thereof for the prevention and treatment of tauopathies.

### BACKGROUND OF THE INVENTION

Tau proteins are abundant in neurons of the central nervous system and play a role in stabilization of microtubules. Pathologies and dementias of the nervous system, such as Alzheimer's disease, are associated with Tau proteins that have become defective and no longer stabilize microtubules properly. Additional neurodegenerative diseases, referred to as tauopathies, are characterized by abundant filamentous Tau. These diseases include tangle-only dementia, chronic traumatic encephalopathy (CTE), argyrophilic grain disease, progressive supranuclear palsy, corticobasal degeneration, globular glial tauopathy, and Pick's disease.

There is a need for the development of Tau-directed therapies for use in the treatment of patients suffering from diseases such as Alzheimer's disease and tauopathies.

### SUMMARY OF THE INVENTION

The present disclosure is directed to individual peptide immunogen constructs targeting portions of the Tau protein for the treatment and/or prevention of tauopathies. The present disclosure is also directed to compositions containing the peptide immunogen constructs, methods of making and using the peptide immunogen constructs, and antibodies produced by the peptide immunogen constructs.

The disclosed peptide immunogen constructs contain about 15 or more amino acids. The peptide immunogen constructs contain a B cell epitope from portions of the longest isoform of the human Tau protein (GenBank: AGF19246.1) having the amino acid sequence of SEQ ID NO: 167 shown in Table 3. The B cell epitope can be linked to a heterologous T helper cell (Th) epitope derived from pathogen proteins through an optional heterologous spacer. The disclosed peptide immunogen constructs stimulate the generation of highly specific antibodies directed against Tau. The disclosed peptide immunogen constructs can be used as an immunotherapy for patients suffering from tauopathies.

The B cell epitope portion of the peptide immunogen constructs have amino acid sequences from the full-length Tau protein (SEQ ID NO: 167). In some embodiments, the B cell epitope has a sequence of or including any one of SEQ ID NOs: 1-8, 36, 39, 40, 48, 50, 51, 52, 57, 58, 59, 62, 87, 88, 129, 49, 35, 99, 135, and 137 as shown in Table 1 or one of Table 4 and FIGS. 13-15.

The peptide immunogen constructs of the present disclosure can contain a heterologous Th epitope amino acid sequence derived from a pathogenic protein (e.g., SEQ ID NOs: 138-166) as shown in Table 2. In some embodiments, the heterologous Th epitope consists of or comprises SEQ ID NO: 151. In some embodiments, the heterologous Th epitope consists of or comprises SEQ ID NO: 149 or 152. In certain embodiments, the heterologous Th epitope is derived from natural pathogens, such as Diphtheria Toxin (SEQ ID NO: 142), Plasmodium Falciparum (SEQ ID NO: 143), Cholera Toxin (SEQ ID NO: 145). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., SEQ ID NOs: 149, 148, or 150).

In some embodiments, the peptide immunogen constructs contain a B cell epitope from Tau linked to a heterologous T helper cell (Th) epitope through an optional heterologous spacer. In certain embodiments, the peptide immunogen constructs contain a B cell antigenic site having or comprising an amino acid sequence from Tau (e.g., any one of SEQ ID NOs: 1-8, 36, 39, 40, 48, 50, 51, 52, 57, 58, 59, 62, 87, 88, 129, 49, 35, 99, 135, and 137) linked to a heterologous Th epitope derived from a pathogenic protein (e.g., SEQ ID NOs: 138-166, e.g., 151, 152, or 149) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the peptide immunogen constructs have the amino acid sequence of one of SEQ ID NOs: 9-16 (e.g., 10 or 15) shown in Table 1 (or a construct in which the positions of the Tau peptide and the Th peptide are reversed).

The present disclosure is also directed to compositions containing A Tau peptide immunogen construct. In some embodiments, the disclosed compositions contain more than one Tau peptide immunogen constructs to cover multiple B epitopes from Tau. In certain embodiments, the compositions contain a mixture of Tau peptide immunogen constructs (e.g., any combination of SEQ ID NOs: 9-16) with more than one heterologous Th epitope derived from pathogenic proteins to cover a broad genetic background in patients. Compositions containing a mixture of peptide immunogen constructs can lead to a higher percentage in responder rate upon immunization for the treatment of tauopathies compared to compositions containing only a single Th peptide immunogen construct.

The present disclosure is also directed to pharmaceutical compositions for the treatment and/or prevention of tauopathies. In some embodiments, the pharmaceutical compositions contain the disclosed peptide immunogen constructs in the form of a stabilized immunostimulatory complex formed through electrostatic associations by mixing a CpG oligomer (e.g., CpG1, CpG2, or CpG3) with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 51 or 720.

The present disclosure is also directed to antibodies directed against the disclosed Tau peptide immunogen constructs. In particular, the peptide immunogen constructs of the present disclosure are able to stimulate the generation of highly specific antibodies that are cross-reactive with the Tau amino acid sequences (e.g., any one of SEQ ID NOs: 1-8, 36, 39, 40, 48, 50, 51, 52, 57, 58, 59, 62, 87, 88, 129, 49, 35, 99, 135, and 137) when administered to a subject. The highly specific antibodies produced by the peptide immunogen constructs are cross reactive with recombinant Tau-containing proteins. The disclosed antibodies bind with high specificity to Tau without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing tauopathies using the disclosed peptide immunogen constructs and/or antibodies directed against the peptide immunogen constructs. In some embodiments, the methods for treating and/or preventing tauopathies including administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to patients with tauopathies. The disclosed methods also include dosing regimens, dosage forms, and routes for administering the peptide immunogen constructs to a host at risk for, or with, tauopathies.

### BRIEF DESCRIPTION OF THE DRAWINGS

FIG. 1 is a schematic diagram showing a Th peptide carrier linked to a target-specific B-cell epitope (Top) and tau primary structure with functional domains and targeting peptides annotated (Bottom).

FIGS. 2A and 2B are a dot blot, set of graphs, and table showing the results of antibody binding characterization.

FIGS. 3A and 3B are a set of graphs showing the results of an aggregation assay using a Tau biosensor line.

FIGS. 4A and 4B are a set of graphs showing the results of a Tau uptake assay.

FIGS. 5A-5C are graphs and photographic images showing the results of vaccination of WT and P301L mice with p5555 kb. The arrowheads in FIG. 6A indicate the injections.

FIG. 6 is a graph showing immunogenicity of p5555 kb and p5187 in rats.

FIGS. 7-10 are a series of graphs showing isotype analysis of antibodies generated in rats.

FIG. 11 is a set of graphs showing the results of a Tau aggregation assay. Guinea pig data are in the left panel and rat data are in the right panel.

FIG. 12 is a set of graphs showing the results of immunogenicity studies in mice.

FIG. 13 shows localization of immunogenic regions within Tau 1-45 through fine epitope mapping.

FIG. 14 shows localization of immunogenic regions within Tau 145-160 through fine epitope mapping.

FIG. 15 shows localization of immunogenic regions within Tau 379-408 through fine epitope mapping.

### DETAILED DESCRIPTION OF THE INVENTION

The present disclosure is directed to individual peptide immunogen constructs targeting portions of the Tau protein for the treatment and/or prevention of Alzheimer's and tauopathies. The present disclosure is also directed to compositions containing the peptide immunogen constructs, methods of making and using the peptide immunogen constructs, and antibodies produced by the peptide immunogen constructs.

The disclosed peptide immunogen constructs contain about 15 or more amino acids. The peptide immunogen constructs contain a B cell epitope from portions of the longest isoform of the human Tau protein (GenBank: AGF19246.1) having the amino acid sequence shown in Table 3. The B cell epitope can be linked to a heterologous T helper cell (Th) epitope derived from pathogen proteins through an optional heterologous spacer. The disclosed peptide immunogen constructs stimulate the generation of highly specific antibodies directed against Tau. The disclosed peptide immunogen constructs can be used as an immunotherapy for patients suffering from Alzheimer's disease and/or tauopathies.

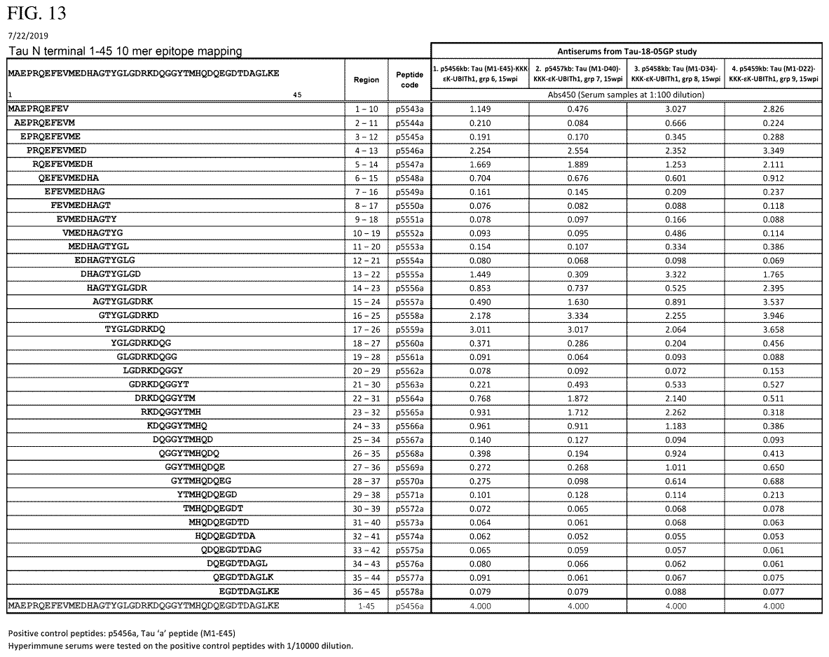

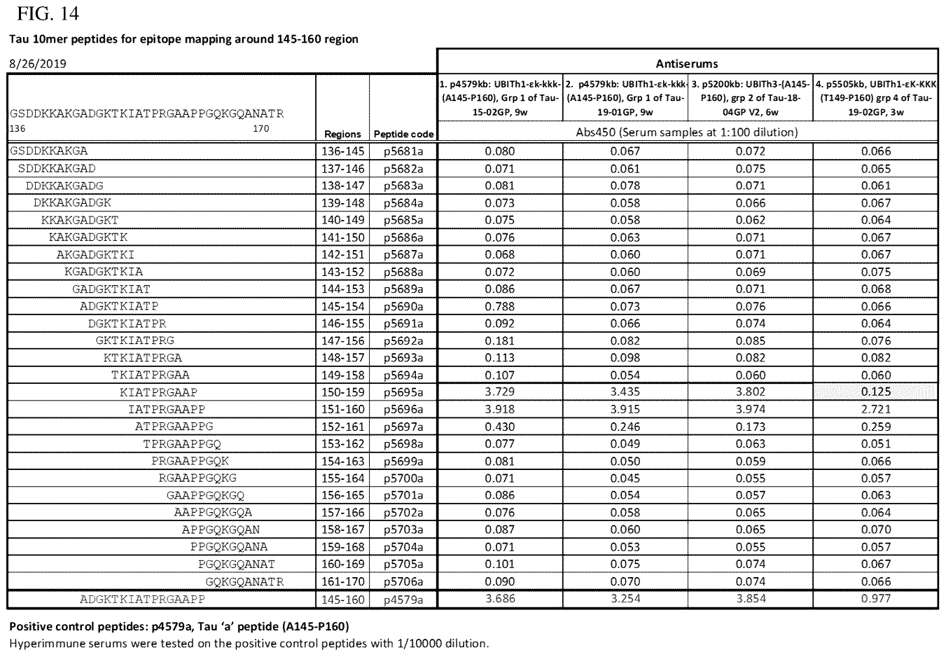

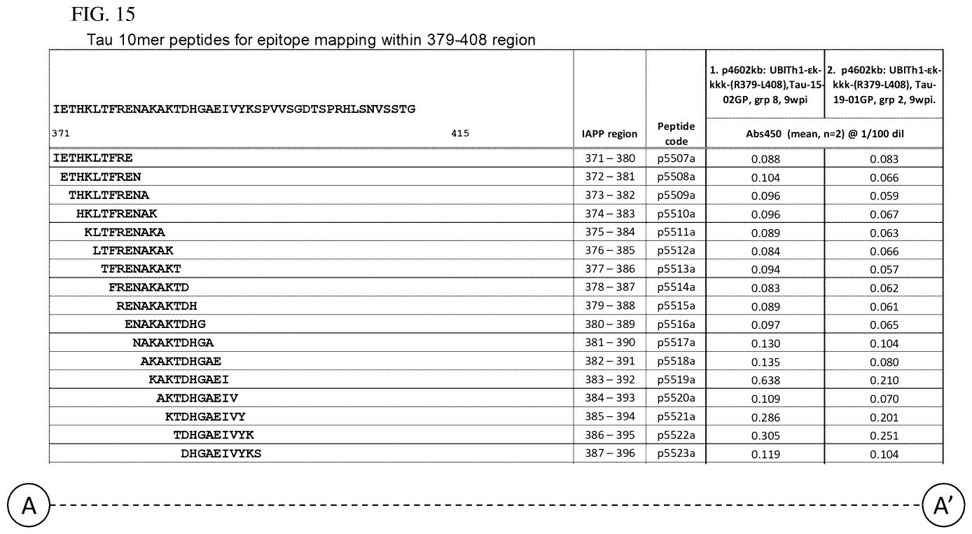

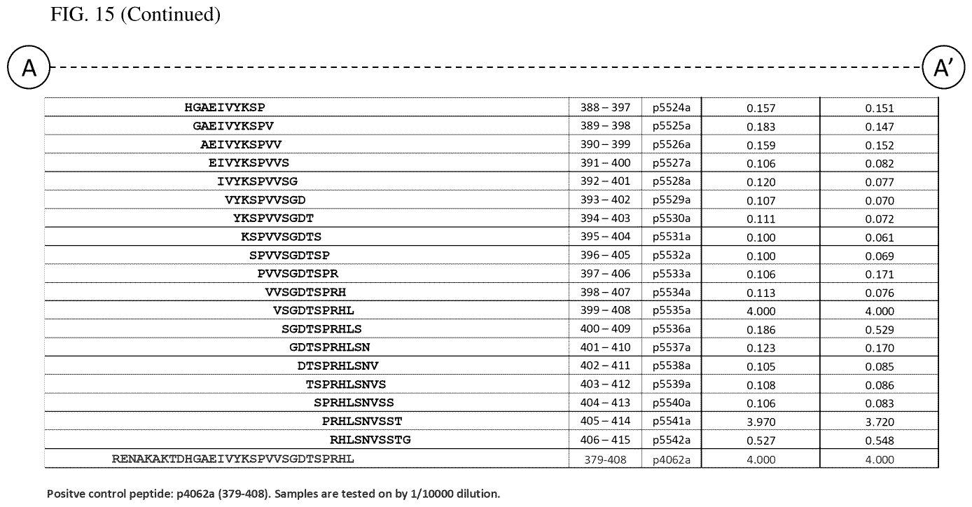

The B cell epitope portion of the peptide immunogen constructs have amino acid sequences from the full-length Tau protein (Table 3). In some embodiments, the B cell epitope has a sequence of or including any one of the target sequences listed in Table 1 (SEQ ID NOs: 1-8). In some embodiments, the B cell epitope has a sequence of or including a sequence of one of Table 4 and FIGS. 13-15 (e.g., any one of SEQ ID NOs: 17-137, e.g., a sequence of p5543a, p5546a, p5547a, p5555a, p5557a, p5558a, p5559a, p5564a, p5565a, p5566a, p5569a, p5695a, p5696, or p5535a, which are SEQ ID NOs: 36, 39, 40, 48, 50, 51, 52, 57, 58, 59, 62, 87, 88, and 129, respectively; or a sequence of p5556a, p5456a, p4579a, p5541a, or p4062a, which are SEQ ID NOs: 49, 35, 99, 135, and 137, respectively).

The peptide immunogen constructs of the present disclosure can contain a heterologous Th epitope amino acid sequence derived from a pathogenic protein (e.g., SEQ ID NOs: 138 to 166) as shown in Table 2. In some embodiments, the Th epitope has or comprises the sequence of SEQ ID NO: 151, 152, or 149. In certain embodiments, the heterologous Th epitope is derived from natural pathogens, such as Diphtheria Toxin (SEQ ID NO: 142), Plasmodium Falciparum (SEQ ID NO: 143), Cholera Toxin (SEQ ID NO: 145). In other embodiments, the heterologous Th epitope is an idealized artificial Th epitope derived from Measles Virus Fusion protein (MVF 1 to 5) or Hepatitis B Surface Antigen (HBsAg 1 to 3) in the form of either single sequence or combinatorial sequences (e.g., SEQ ID NOs: 149, 148, or 150).

In some embodiments, the peptide immunogen constructs contain a B cell epitope from Tau linked to a heterologous T helper cell (Th) epitope through an optional heterologous spacer. In certain embodiments, the peptide immunogen constructs contain a B cell antigenic site having or comprising an amino acid sequence from Tau (see, e.g., Table 1 (one of SEQ ID NOs: 1-8) and Table 4 and FIGS. 13-15 (e.g., one of SEQ ID NOs: 36, 39, 40, 48, 50, 51, 52, 57, 58, 59, 62, 87, 88, 129, 49, 35, 99, 135, and 137) linked to a heterologous Th epitope derived from a pathogenic protein (e.g., SEQ ID NOs: 138 to 166, e.g., 151, 152, or 149) through an optional heterologous spacer. In some embodiments, the optional heterologous spacer is a molecule or chemical structure capable of linking two amino acids and/or peptides together. In certain embodiments, the spacer is a naturally occurring amino acid, a non-naturally occurring amino acid, or a combination thereof. In specific embodiments, the peptide immunogen constructs have an amino acid sequence shown in Table 1 (e.g., a sequence of any one of SEQ ID NOs: 9-16 (e.g., 10 or 15) or a construct in which the positions of the Tau peptides and the Th epitopes are reversed).

The present disclosure is also directed to compositions containing a Tau peptide immunogen construct. In some embodiments, the disclosed compositions contain more than one Tau peptide immunogen constructs to cover multiple B epitopes from Tau. In certain embodiments, the compositions contain a mixture of Tau peptide immunogen constructs with more than one heterologous Th epitope derived from pathogenic protein to cover a broad genetic background in patients. Compositions containing a mixture of peptide immunogen constructs can lead to a higher percentage in responder rate upon immunization for the treatment of Alzheimer's disease and/or tauopathies compared to compositions containing only a single peptide immunogen construct.

The present disclosure is also directed to pharmaceutical compositions for the treatment and/or prevention of Alzheimer's disease and/or tauopathies. In some embodiments, the pharmaceutical compositions contain the disclosed peptide immunogen constructs in the form of a stabilized immunostimulatory complex formed through electrostatic associations by mixing a CpG oligomer with a composition containing a peptide immunogen complex. Such stabilized immunostimulatory complexes are able to further enhance the immunogenicity of the peptide immunogen constructs. In some embodiments, the pharmaceutical compositions contain adjuvants such as mineral salts, including alum gel (ALHYDROGEL), aluminum phosphate (ADJUPHOS), or water-in-oil emulsions including MONTANIDE ISA 51 or 720.

The present disclosure is also directed to antibodies directed against the disclosed Tau peptide immunogen constructs. In particular, the peptide immunogen constructs of the present disclosure are able to stimulate the generation of highly specific antibodies that are cross-reactive with the Tau amino acid sequences when administered to a subject. The highly specific antibodies produced by the peptide immunogen constructs are cross reactive with recombinant Tau-containing proteins. The disclosed antibodies bind with high specificity to Tau without much, if any, directed to the heterologous Th epitopes employed for immunogenicity enhancement, which is in sharp contrast to the conventional protein or other biological carriers used for such peptide antigenicity enhancement.

The present disclosure also includes methods for treating and/or preventing Alzheimer's disease and/or tauopathies using the disclosed peptide immunogen constructs and/or antibodies directed against the peptide immunogen constructs. In some embodiments, the methods for treating and/or preventing Alzheimer's disease and/or tauopathies including administering to a host a composition containing a disclosed peptide immunogen construct. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to patients with Alzheimer's disease and/or tauopathies. The disclosed methods also include dosing regimens, dosage forms, and routes for administering the peptide immunogen constructs to a host at risk for, or with, Alzheimer's disease and/or tauopathies.

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Tau Peptide Immunogen Construct

The present disclosure provides peptide immunogen constructs containing a B cell epitope with an amino acid sequence from Tau covalently linked to a heterologous T helper cell (Th) epitope directly or through an optional heterologous spacer.

The phrase “Tau peptide immunogen construct” or “peptide immunogen construct,” as used herein, refers to a peptide containing (a) a B cell epitope having about 8 or more amino acid residues from the full-length sequence of the longest Tau isoform (SEQ ID NO: 167) (e.g., about 8-40, 9-30, 10-25, 8-20, 9-18, or 10-15; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids); (b) a heterologous Th epitope; and (c) an optional heterologous spacer.

In certain embodiments, the Tau peptide immunogen construct can be represented by the formulae:

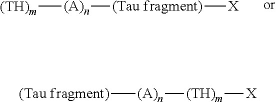

wherein
Th is a heterologous T helper epitope;
A is a heterologous spacer;
(Tau fragment) is a B cell epitope having about 8 to about 40 amino acid residues from SEQ ID NO: 167 (e.g., about 8-40, 9-30, 10-25, 8-20, 9-18, or 10-15; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
X is an α-COOH or α-CONH₂ of an amino acid;
m is from 1 to about 4; and
n is from 0 to about 10.

The various components of the disclosed Tau peptide immunogen construct are described below.

a. Tau Fragments

The disclosed peptide immunogen constructs contain about 8 or more amino acids. The peptide immunogen constructs contain a B cell epitope from portions of the longest isoform of the human Tau protein (GenBank: AGF19246.1) having the amino acid sequence of SEQ ID NO: 167 shown in Table 3. The B cell epitope can be linked to a heterologous T helper cell (Th) epitope derived from pathogen proteins through an optional heterologous spacer. The disclosed peptide immunogen constructs stimulate the generation of highly specific antibodies directed against Tau. The disclosed peptide immunogen constructs can be used as an immunotherapy for patients suffering from Alzheimer's disease and/or tauopathies.

The B cell epitope portion of the peptide immunogen constructs have amino acid sequences from the full-length Tau protein (SEQ ID NO: 167). In some embodiments, the B cell epitope has or comprises a sequence shown in Table 1 (one of SEQ ID NOs: 1-8) or in Table 4 and FIGS. 13-15 (e.g., one of SEQ ID NOs: 36, 39, 40, 48, 50, 51, 52, 57, 58, 59, 62, 87, 88, 129, 49, 35, 99, 135, and 137).

In some embodiments, the Tau peptide of the peptide immunogen constructs comprises or consists of a peptide of any one of Tables 1, 4 and FIGS. 13-15.

In some embodiments, the Tau peptide of the peptide immunogen constructs comprises or consists of a peptide of any one of Tables 1, 4 and FIGS. 13-15, wherein the peptide includes 1, 2, 3, 4, or 5 additional amino acids of the Tau protein of SEQ ID NO: 167 on the N-terminal end of the peptide of Tables 1, 4 and FIGS. 13-15. In some embodiments, the Tau peptide of the peptide immunogen constructs comprises or consists of a peptide of any one of Tables 1, 4 and FIGS. 13-15, wherein the peptide includes 1, 2, 3, 4, or 5 additional amino acids of the Tau protein of SEQ ID NO: 167 on the C-terminal end of the peptide of Tables 1, 4 and FIGS. 13-15. In some embodiments, the Tau peptide of the peptide immunogen constructs comprises or consists of a peptide of any one of Tables 1, 4 and FIGS. 13-15, wherein the peptide includes 1, 2, 3, 4, or 5 additional amino acids of the Tau protein of SEQ ID NO: 167 on the N-terminal end and/or the C-terminal end of the peptide of Tables 1, 4 and FIGS. 13-15. The additional amino acids in the latter peptides are to be understood to be those of the Tau protein of SEQ ID NO: 167 that are contiguous with the peptide sequences of Tables 1, 4 and FIGS. 13-15.

In some embodiments, the Tau peptide of the peptide immunogen constructs comprises or consists of about 8 to about 40 amino acid residues from SEQ ID NO: 167 (e.g., about 8-40, 9-30, 10-25, 8-20, 9-18, or 10-15; or about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids). In some embodiments, the Tau peptide of the peptide immunogen constructs are or comprise any one of the peptides of Tables 1, 4 and FIGS. 13-15, e.g., any one of SEQ ID NOs: 1-8, 36, 39, 40, 48, 50, 51, 52, 57, 58, 59, 62, 87, 88, 129, 49, 35, 99, 135, and 137, wherein optionally the peptide has one or more of the additional amino acids noted above.

b. Heterologous T Helper Cell Epitopes (Th Epitopes)

The present disclosure provides peptide immunogen constructs containing a B cell epitope from Tau covalently linked to a heterologous T helper cell (Th) epitope directly or through an optional heterologous spacer.

The heterologous Th epitope in the Tau peptide immunogen construct enhances the immunogenicity of the Tau fragments, which facilitates the production of specific high titer antibodies directed against the optimized target B cell epitope (i.e., the Tau fragment) through rational design.

The term “heterologous”, as used herein, refers to an amino acid sequence that is derived from an amino acid sequence that is not part of, or homologous with, the wild-type sequence of Tau. Thus, a heterologous Th epitope is a Th epitope derived from an amino acid sequence that is not naturally found in Tau (i.e., the Th epitope is not autologous to Tau). Since the Th epitope is heterologous to Tau, the natural amino acid sequence of Tau is not extended in either the N-terminal or C-terminal directions when the heterologous Th epitope is covalently linked to the Tau fragment.

The heterologous Th epitope of the present disclosure can be any Th epitope that does not have an amino acid sequence naturally found in Tau. The Th epitope can have an amino acid sequence derived from any species (e.g., human, pig, cattle, dog, rat, mouse, guinea pigs, etc.). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the Tau peptide immunogen constructs will be directed towards the Th epitope, thus allowing a very focused immune response directed to the targeted B cell epitope of the Tau fragment.

Th epitopes of the present disclosure include, but are not limited to, amino acid sequences derived from foreign pathogens, as exemplified in Table 2 (SEQ ID NOs: 138 to 166, e.g., 151, 152, or 149). Further, Th epitopes include idealized artificial Th epitopes and combinatorial idealized artificial Th epitopes (e.g., SEQ ID NOs: 139 and 146-152). The heterologous Th epitope peptides presented as a combinatorial sequence (e.g., SEQ ID NOs: 147-150), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 147-150 which are shown in Table 2. Th epitope peptides of the present invention provide broad reactivity and immunogenicity to animals and patients from genetically diverse populations.

Tau peptide immunogen constructs comprising Th epitopes are produced simultaneously in a single solid-phase peptide synthesis in tandem with the Tau fragment. Th epitopes also include immunological analogues of Th epitopes. Immunological Th analogues include immune-enhancing analogs, cross-reactive analogues and segments of any one of these Th epitopes that are sufficient to enhance or stimulate an immune response to the Tau fragments.

Functional immunologically analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the Tau fragments. Table 2 identifies another variation of a functional analogue for Th epitope peptide. In particular, SEQ ID NOs: 139 and 146 of MvF1 and MvF2 Th are functional analogues of SEQ ID NOs: 149 and 151 of MvF4 and MvF5 in that they differ in the amino acid frame by the deletion (SEQ ID NOs: 139 and 146) or the inclusion (SEQ ID NOs: 149 and 151) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 139, 146, 147, 149, and 151) and from Hepatitis Surface protein HBsAg 1-3 Ths (SEQ ID NOs: 148, 150, and 152).

The Th epitope in the Tau peptide immunogen construct can be covalently linked at either N- or C-terminal end of the Tau peptide. In some embodiments, the Th epitope is covalently linked to the N-terminal end of the Tau peptide. In other embodiments, the Th epitope is covalently linked to the C-terminal end of the Tau peptide. In certain embodiments, more than one Th epitope is covalently linked to the Tau fragment. When more than one Th epitope is linked to the Tau fragment, each Th epitope can have the same amino acid sequence or different amino acid sequences. In addition, when more than one Th epitope is linked to the Tau fragment, the Th epitopes can be arranged in any order. For example, the Th epitopes can be consecutively linked to the N-terminal end of the Tau fragment, or consecutively linked to the C-terminal end of the Tau fragment, or a Th epitope can be covalently linked to the N-terminal end of the Tau fragment while a separate Th epitope is covalently linked to the C-terminal end of the Tau fragment. There is no limitation in the arrangement of the Th epitopes in relation to the Tau fragment. For the immunogen constructs depicted in Table 1, it should be understood that the disclosure includes corresponding immunogenic constructs in which the positions of the Tau peptide and the Th epitope are reversed from the orientation set forth in Table 1.

In some embodiments, the Th epitope is covalently linked to the Tau fragment directly. In other embodiments, the Th epitope is covalently linked to the Tau fragment through a heterologous spacer described in further detail below.

c. Heterologous Spacer

The disclosed Tau peptide immunogen constructs optionally contain a heterologous spacer that covalently links the B cell epitope from Tau to the heterologous T helper cell (Th) epitope.

As discussed above, the term “heterologous,” refers to an amino acid sequence that is derived from an amino acid sequence that is not part of, or homologous with, the wild-type sequence of Tau. Thus, the natural amino acid sequence of Tau is not extended in either the N-terminal or C-terminal directions when the heterologous spacer is covalently linked to the B cell epitope from Tau because the spacer is heterologous to the Tau sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the Tau peptide immunogen construct. Structurally, the spacer provides a physical separation of the Th epitope from the B cell epitope of the Tau fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 168), where Xaa is any amino acid, and preferably aspartic acid.

The spacer can also provide functional features to the Tau peptide immunogen construct. For example, the spacer can be designed to change the overall charge of the Tau peptide immunogen construct, which can affect the solubility of the peptide immunogen construct. Additionally, changing the overall charge of the Tau peptide immunogen construct can affect the ability of the peptide immunogen construct to associate with other compounds and reagents. As discussed in further detail below, the Tau peptide immunogen construct can be formed into a stable immunostimulatory complex with a highly charged oligonucleotide, such as CpG oligomers through electrostatic association. The overall charge of the Tau peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, ß-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the Tau peptide immunogen construct can be covalently linked at either N- or C-terminal end of the Th epitope and the Tau peptide. In some embodiments, the spacer is covalently linked to the C-terminal end of the Th epitope and to the N-terminal end of the Tau peptide. In other embodiments, the spacer is covalently linked to the C-terminal end of the Tau peptide and to the N-terminal end of the Th epitope. In certain embodiments, more than one spacer can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the Tau fragment.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N) Lys, or ε-N-Lys-Lys-Lys-Lys (SEQ ID NO: 169).

d. Specific Embodiments of the Tau Peptide Immunogen Construct

The Tau peptide immunogen construct can be represented by the formulae:

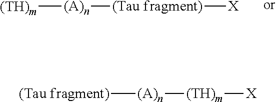

wherein
Th is a heterologous T helper epitope;
A is a heterologous spacer;
(Tau fragment) is a B cell epitope having about 8 to about 40 amino acid residues from the full-length Tau protein of SEQ ID NO: 167 (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
X is an α-COOH or α-CONH₂ of an amino acid;
m is from 1 to about 4; and
n is from 0 to about 10.

In certain embodiments, the heterologous Th epitope in the Tau peptide immunogen construct has an amino acid sequence selected from any one of those set forth in Table 2. In certain embodiments, the Tau peptide immunogen construct contains more than one Th epitope.

In certain embodiments, the optional heterologous spacer is selected from any one of Lys-, Gly-, Lys-Lys-Lys-, (α, ε-N) Lys, ε-N-Lys-Lys-Lys-Lys (SEQ ID NO: 169), and combinations thereof. In specific embodiments, the heterologous spacer is-N-Lys-Lys-Lys-Lys (SEQ ID NO: 169).

In certain embodiments, the Tau fragment has about 8 to about 40 (e.g., 10-30, 10-25, 12-20, or 12-15) amino acid residues from the full-length Tau protein of SEQ ID NO: 167. In specific embodiments, the Tau fragment has an amino acid sequence shown in Table 1 (one of SEQ ID NOs: 1-8) or in one of Table 4 and FIGS. 13-15 (e.g., one of SEQ ID NOs: 36, 39, 40, 48, 50, 51, 52, 57, 58, 59, 62, 87, 88, 129, 49, 35, 99, 135, and 137).

In some embodiments, the tau peptide immunogen construct is selected from the group consisting of:

a.
SEQ ID NO: 9
MAEPRQEFEVMEDHAGTYGLGDKKK-eK-ISITEIKGVIVHRIETI
LF-NH2;
b.
SEQ ID NO: 10
DHAGTYGLGDKKK-eK-ISITEIKGVIVHRIETILF-NH2;
c.
SEQ ID NO: 11
ISITEIKGVIVHRIETILF-eK-KKKNITHVPGGGNKK-NH2;
d.
SEQ ID NO: 12
ISITEIKGVIVHRIETILF-eK-KKKKDNIKHVPGGGSVQIVYK-NH2;
e.
SEQ ID NO: 13
ISITEIKGVIVHRIETILF-eK-KKKTKIATPRGAAPP-NH2;
f.
SEQ ID NO: 14
ISITEIKGVIVHRIETILF-eK-KKKVVRTPPKSPSSAKSRL-NH2;
g.
SEQ ID NO: 15
ISITEIKGVIVHRIETILF-eK-KKKIKHVPGGGSVQIVYK-NH2;
and
h.
SEQ ID NO: 16
ISITEIKGVIVHRIETILF-eK-KKKVSGDTSPRHLSNVSST-NH2

In some embodiments, the T cell epitope of any one of SEQ ID NOs: 9-16 (e.g., 10 or 15) is replaced with a T cell epitope of any one of SEQ ID NOs: 138-166. In some embodiments, the positions of the B cell epitope and the T cell epitope of any one of SEQ ID NOs: 9-16 (e.g., 10 or 15) is reversed within the molecule (i.e., if the B cell epitope is at the N-terminal end of the original molecule, it is placed at the C-terminal end of the molecule in the reversed molecule, etc.).

a. Variants, Homologues, and Functional Analogues

Variants and analogs of the above immunogenic peptides that induce and/or cross-react with antibodies to the preferred epitopes of Tau protein can also be used. Analogs, including allelic, species, and induced variants, typically differ from naturally occurring peptides at one, two, or a few positions, often by virtue of conservative substitutions. Analogs typically exhibit at least 80 or 90% sequence identity with natural peptides. Some analogs also include unnatural amino acids or modifications of N- or C-terminal amino acids at one, two, or a few positions.

Variants that are functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Variants also include variations to the phosphorylated residues. For example, variants can include different residues within the peptides that are phosphorylated. Variant immunogenic Tau peptides can also include pseudo-phosphorylated peptides. The pseudo-phosphorylated peptides are generated by substituting one or more of the phosphorylated serine, threonine, and tyrosine residues of the Tau peptides with acidic amino acid residues such as glutamic acid and aspartic acid.

### Compositions

The present disclosure also provides compositions comprising the disclosed Tau immunogen construct.

a. Peptide Compositions

Compositions containing a disclosed Tau peptide immunogen construct can be in liquid or solid form. Liquid compositions can include water, buffers, solvents, salts, and/or any other acceptable reagent that does not alter the structural or functional properties of the Tau peptide immunogen construct. Peptide compositions can contain one or more of the disclosed Tau peptide immunogen constructs.

b. Pharmaceutical Compositions

The present disclosure is also directed to pharmaceutical compositions containing the disclosed Tau peptide immunogen construct.

Pharmaceutical compositions can contain carriers and/or other additives in a pharmaceutically acceptable delivery system. Accordingly, pharmaceutical compositions can contain a pharmaceutically effective amount of a Tau peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the Tau peptide immunogen construct without having any specific antigenic effect itself. Adjuvants used in the pharmaceutical composition can include oils, aluminum salts, virosomes, aluminum phosphate (e.g., ADJU-PHOS®), aluminum hydroxide (e.g., ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), Tween® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with Emulsigen or Emulsigen D as the adjuvant.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the Tau peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can also be formulated in a suitable dosage unit form. In some embodiments, the pharmaceutical composition contains from about 0.5 μg to about 1 mg of the Tau peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one Tau peptide immunogen construct. A pharmaceutical composition containing a mixture of more than one Tau peptide immunogen construct to allow for synergistic enhancement of the immunoefficacy of the constructs. Pharmaceutical compositions containing more than one Tau peptide immunogen construct can be more effective in a larger genetic population due to a broad MHC class II coverage thus provide an improved immune response to the Tau peptide immunogen constructs.

In some embodiments, the pharmaceutical composition contains a Tau peptide immunogen construct shown in Table 1, as well as homologues, analogues and/or combinations thereof. In some embodiments, pharmaceutical compositions contain a Tau peptide immunogen construct including a Tau peptide from one of Tables 1, 4 and FIGS. 13-15.

Pharmaceutical compositions containing a Tau peptide immunogen construct can be used to elicit an immune response and produce antibodies in a host upon administration.

c. Immunostimulatory Complexes

The present disclosure is also directed to pharmaceutical compositions containing a Tau peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in the form of a particulate, which can efficiently present the Tau peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the Tau peptide immunogen to the cells of the immune system of a host following parenteral administration.

The stabilized immunostimulatory complex can be formed by complexing a Tau peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the Tau peptide immunogen construct is designed to contain a cationic portion that is positively charged at a pH in the range of 5.0 to 8.0. The net charge on the cationic portion of the Tau peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the Tau peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the Tau peptide immunogen construct is the heterologous spacer. In certain embodiments, the cationic portion of the Tau peptide immunogen construct has a charge of +4 when the spacer sequence is (α, ε-N) Lys, ε-N-Lys-Lys-Lys-Lys (SEQ ID NO: 169).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T.

In some embodiments, the CpG oligonucleotide is as described in WO 03/068169, the contents of which are incorporated by reference. In some embodiments, the CpG comprises or consists of a sequence of CpG1 (tcgtcgtttt gtcgttttgt cgttttgtcg tt; SEQ ID NO: 170), CpG2 (tcgtcgtttt gtcgttttgt cgtt; SEQ ID NO: 171), or CpG3 (tcgtcgtttt gtogttttgt cgtt; SEQ ID NO: 172). In some embodiments, the CpG oligonucleotide (e.g., CpG1, CpG2, or CpG3) is a phosphorothioate oligonucleotide.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Antibodies

The present disclosure also provides antibodies elicited by the Tau peptide immunogen construct.

The disclosed Tau peptide immunogen constructs, comprising a Tau fragment, heterologous Th epitope, and optional heterologous spacer, are capable of eliciting an immune response and the production of antibodies when administered to a host. The design of the Tau peptide immunogen constructs can break tolerance to self Tau and elicit the production of site-specific antibodies that recognize conformational, not linear, epitopes.

The antibodies produced by the Tau peptide immunogen constructs recognize and bind to Tau in the forms of monomers, dimers, trimers, and oligomers.

Antibodies elicited by the Tau peptide immunogen constructs surprisingly can prevent aggregation of Tau (anti-aggregation activity) and can disassociate preformed Tau aggregates (disaggregation activity).

The resulting immune responses from animals immunized with Tau peptide immunogen constructs of the present invention demonstrated the ability of the constructs to produce potent site-directed antibodies that are reactive with Tau in the forms of monomers, dimers, trimers, and oligomers.

### Methods

The present disclosure is also directed to methods for making and using the Tau peptide immunogen constructs, compositions, and pharmaceutical compositions.

a. Methods for Manufacturing the Tau Peptide Immunogen Construct

The Tau peptide immunogen constructs of this disclosure can be made by chemical synthesis methods well known to the ordinarily skilled artisan (see, e.g., Fields et al., Chapter 3 in Synthetic Peptides: A User's Guide, ed. Grant, W. H. Freeman & Co., New York, NY, 1992, p. 77). The Tau peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of Tau peptide immunogen constructs comprising combinatorial library peptides for Th epitopes can be accomplished by providing a mixture of alternative amino acids for coupling at a given variable position.

After complete assembly of the desired Tau peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art. The quality of peptides produced by this chemical process can be controlled and defined and, as a result, reproducibility of Tau peptide immunogen constructs, immunogenicity, and yield can be assured.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The Tau peptide immunogen constructs can also be made using recombinant DNA technology including nucleic acid molecules, vectors, and/or host cells. As such, nucleic acid molecules encoding the Tau peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the Tau peptide immunogen construct and immunologically functional analogues thereof. For example, methods can include a step of incubating a host cell containing an expression vector containing a nucleic acid molecule encoding a Tau peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the Immunostimulatory complexes comprising Tau peptide immunogen constructs and CpG oligodeoxynucleotide (ODN) molecule. Stabilized immunostimulatory complexes (ISC) are derived from a cationic portion of the Tau peptide immunogen construct and a polyanionic CpG ODN molecule. The self-assembling system is driven by electrostatic neutralization of charge. Stoichiometry of the molar charge ratio of cationic portion of the Tau peptide immunogen construct to anionic oligomer determines extent of association. The non-covalent electrostatic association of Tau peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This novel particulate system comprising CpG ODN and Tau fragment derived peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing Tau peptide immunogen constructs. In certain embodiments, the pharmaceutical compositions employ water in oil emulsions and in suspension with mineral salts.

In order for a pharmaceutical composition to be used by a large population and with prevention of Tau aggregation also being part of the goal for administration, safety becomes another important factor for consideration. Despite the use of water-in-oil emulsions in humans for many formulations in clinical trials, Alum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-AI-D-isoglu-L-Ala-dipalmitoxy propylamide (DTP-DPP) Theramide™), or other bacterial cell wall components. Oil-in-water emulsions include MF59 (see WO 90/14837 to Van Nest et al., which is hereby incorporated by reference in its entirety), containing 5% Squalene, 0.5% Tween 80, and 0.5% Span 85 (optionally containing various amounts of MTP-PE) formulated into submicron particles using a microfluidizer; SAF, containing 10% Squalene, 0.4% Tween 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% Tween 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), granulocyte-macrophage colony stimulating factor (GM-CSF), and tumor necrosis factor (TNF).

The choice of an adjuvant depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, alum, MPL or Incomplete Freund's adjuvant (Chang et al., Advanced Drug Delivery Reviews 32:173-186 (1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods Using Pharmaceutical Compositions

The present disclosure also includes methods of using pharmaceutical compositions containing Tau peptide immunogen constructs.

In certain embodiments, the pharmaceutical compositions containing Tau peptide immunogen constructs can be used for:
(a) inhibiting Tau aggregation in a host;
(b) inducing disaggregate of preformed Tau aggregates in a host;
(c) reducing neurodegeneration triggered by exogeneous Tau aggregates in a host;
(d) reducing neurodegeneration in Tau overexpressing cells;
(e) reducing serum Tau levels in a host;
(f) reducing oligomeric Tau level in the brain of a host;
(g) reducing neuropathology and recovery of motor activities in a host; and the like.

The above methods comprise administering a pharmaceutical composition comprising a pharmacologically effective amount of a Tau peptide immunogen construct to a host in need thereof.

As used herein a “tauopathy” encompasses any neurodegenerative disease that involves the pathological aggregation of the microtubule protein Tau within the brain. Accordingly, in addition to both familial and sporadic Alzheimer's disease, other tauopathies that can be treated using the methods of the present invention include, without limitation, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy. The methods can further be used in the prevention and treatment of Lewy body disease.

Another aspect of the present disclosure is directed to a method of promoting clearance of Tau aggregates from the brain of a subject. This method involves administering, to the subject, any one or more immunogenic Tau peptides of one of Tables 1, 4 and FIGS. 13-15, or one or more antibodies recognizing an epitope of one of such peptides, under conditions effective to promote clearance of Tau aggregates from the brain of the subject.

The clearance of Tau aggregates includes clearance of neurofibrillary tangles and/or the pathological Tau precursors to neurofibrillary tangles. Neurofibrillary tangles are often associated with neurodegenerative diseases including, for example, sporadic and familial Alzheimer's disease, amyotrophic lateral sclerosis, argyrophilic grain dementia, dementia pugilistica, chronic traumatic encephalopathy, diffuse neurofibrillary tangles with calcification, Down syndrome, Gerstmann-Straussler-Scheinker disease, Hallervorden-Spatz disease, hereditary frontotemporal dementia, parkinsonism linked to chromosome 17 (FTDP-17), inclusion body myositis, Creutsfeld-Jakob disease, multiple system atrophy, Niemann-Pick disease type C, Pick's disease, prion protein cerebral amyloid angiopathy, sporadic corticobasal degeneration, progressive supranuclear palsy, subacute sclerosing panencephalitis, myotonic dystrophy, motor neuron disease with neurofibrillary tangles, tangle only dementia, and progressive subcortical gliosis.

Another aspect of the present disclosure is directed to a method of slowing the progression of a Tau-pathology related behavioral phenotype in a subject. This method involves administering, to the subject, any one or more immunogenic Tau peptides of one of Tables 1, 4 and FIGS. 13-15, or one or more antibodies recognizing an immunogenic Tau epitope of one of such peptides, under conditions effective to slow the Tau-pathology related behavioral phenotype in the subject.

As used herein, a Tau-pathology related behavioral phenotype includes, without limitation, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

In accordance with the methods of the present disclosure, in one embodiment, an immunogenic Tau peptide or a combination of immunogenic Tau peptides are administered to a subject in need. Suitable immunogenic Tau peptide fragments of the Tau protein contain one or more antigenic epitopes that mimic the pathological form of the Tau protein. Exemplary immunogenic Tau epitopes are phosphorylated at one or more amino acids that are phosphorylated in the pathological form of Tau, but not phosphorylated in the normal or non-pathological form of Tau.

In some embodiments, administration of an immunogenic Tau peptide induces an active immune response in the subject to the immunogenic Tau peptide and to the pathological form of Tau, thereby facilitating the clearance of related Tau aggregates, slowing the progression of Tau-pathology related behavior and treating the underlying tauopathy. In accordance with this aspect of the present invention, an immune response involves the development of a beneficial humoral (antibody mediated) and/or a cellular (mediated by antigen-specific T cells or their secretion products) response directed against the immunogenic Tau peptide.

The presence of a humoral immunological response can be determined and monitored by testing a biological sample (e.g., blood, plasma, serum, urine, saliva feces, CSF or lymph fluid) from the subject for the presence of antibodies directed to the immunogenic Tau peptide. Methods for detecting antibodies in a biological sample are well known in the art, e.g., ELISA, Dot blots, SDS-PAGE gels or ELISPOT. The presence of a cell-mediated immunological response can be determined by proliferation assays (CD4+ T cells) or CTL (cytotoxic T lymphocyte) assays which are readily known in the art.

Isolated immunogenic Tau peptides of the present invention are, include, or comprise any one of the amino acid sequences of Tables 1, 4 and FIGS. 13-15 below. In preferred embodiments, the peptides of Table 4 and FIGS. 13-15 are those that have at least one value that is greater than 1.

### SPECIFIC EMBODIMENTS

(1) A Tau peptide immunogen construct can be represented by the formulae:

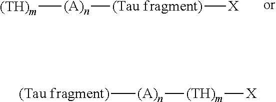

wherein
Th is a heterologous T helper epitope;
A is a heterologous spacer;
(Tau fragment) is a B cell epitope having about 8 to about 40 amino acid residues from the full-length Tau protein of SEQ ID NO: 167 (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
X is an α-COOH or α-CONH₂ of an amino acid;
m is from 1 to about 4; and
n is from 0 to about 10.

(2) The Tau peptide immunogen construct according to (1), wherein the Tau fragment is selected from the group consisting of SEQ ID NOs: 1-8, 36, 39, 40, 48, 50, 51, 52, 57, 58, 59, 62, 87, 88, 129, 49, 35, 99, 135, and 137.

(3) The Tau peptide immunogen construct according to any one of (1) or (2), wherein the Th epitope is selected from the group consisting of SEQ ID NOs: 138-166, e.g., 151, 152, or 149.

(4) The Tau peptide immunogen construct according to any one of (1) to (3), wherein the peptide immunogen construct is selected from the group consisting of SEQ ID NOs: 9-16 (e.g., 10 or 15) (or constructs in which the positions of the Tau peptide and the Th epitope are reversed).

(5) A Tau peptide immunogen construct comprising:
a B cell epitope comprising about 8 to about 40 amino acid residues from the full-length Tau protein sequence of SEQ ID NO: 167 (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids);
a T helper epitope comprising an amino acid sequence selected from the group consisting of SEQ ID NOS: 138-166; and
an optional heterologous spacer selected from the group consisting of an amino acid, Lys-, Gly-, Lys-Lys-Lys-, (α, ε-N) Lys, and ε-N-Lys-Lys-Lys-Lys (SEQ ID NO: 169),
wherein the B cell epitope is covalently linked to the T helper epitope directly or through the optional heterologous spacer.

(6) The Tau peptide immunogen construct of (5), wherein the B cell epitope is selected from the group consisting of SEQ ID NOs: 1-8, 36, 39, 40, 48, 50, 51, 52, 57, 58, 59, 62, 87, 88, 129, 49, 35, 99, 135, and 137.

(7) The Tau peptide immunogen construct of (5) or (6), wherein the T helper epitope is selected from the group consisting of SEQ ID NOs: 138-166.

(8) The Tau peptide immunogen construct of any one of (5) to (7), wherein the optional heterologous spacer is (a, ε-N) Lys or ε-N-Lys-Lys-Lys-Lys (SEQ ID NO: 169).

(9) The Tau peptide immunogen construct of any one of (5) to (8), wherein the T helper epitope is covalently linked to the amino terminus of the B cell epitope.

(10) The Tau peptide immunogen construct of any one of (5) to (8), wherein the T helper epitope is covalently linked to the amino terminus of the B cell epitope through the optional heterologous spacer.

(11) A composition comprising a Tau peptide immunogen construction according to any one of (1) to (10).

(12) A pharmaceutical composition comprising:
a. a peptide immunogen construct according to any one of (1) to (10); and
b. and a pharmaceutically acceptable delivery vehicle and/or adjuvant.

(13) The pharmaceutical composition of (12), wherein
a. the Tau peptide immunogen construct is selected from the group consisting of SEQ ID NOs: 9-16 or a Tau peptide immunogen construct in which the positions of the B cell epitope and the T cell epitope are reversed as compared to one of SEQ ID NOs: 9-16 (e.g., 10 or 15); and
b. the Tau peptide immunogen construct is mixed with an CpG oligodeoxynucleotide (ODN), e.g., CpG1, CpG2, or CpG3, to form a stabilized immunostimulatory complex.

(14) An isolated antibody or epitope-binding fragment thereof that specifically binds to the B cell epitope of the Tau peptide immunogen construct according to any one of (1) to (10).

(15) The isolated antibody or epitope-binding fragment thereof according to (14) bound to the Tau peptide immunogen construct.

(16) A composition comprising the isolated antibody or epitope-binding fragment thereof according to (14) or 15).

(17) A Tau peptide immunogen construct selected from the group consisting of:

a.
SEQ ID NO: 9
MAEPRQEFEVMEDHAGTYGLGDKKK-eK-ISITEIKGVIVHRIETI
LF-NH2;
b.
SEQ ID NO: 10
DHAGTYGLGDKKK-eK-ISITEIKGVIVHRIETILF-NH2;
c.
SEQ ID NO: 11
ISITEIKGVIVHRIETILF-eK-KKKNITHVPGGGNKK-NH2;
d.
SEQ ID NO: 12
ISITEIKGVIVHRIETILF-eK-KKKKDNIKHVPGGGSVQIVYK-NH2;
e.
SEQ ID NO: 13
ISITEIKGVIVHRIETILF-eK-KKKTKIATPRGAAPP-NH2;
f.
SEQ ID NO: 14
ISITEIKGVIVHRIETILF-eK-KKKVVRTPPKSPSSAKSRL-NH2;
g.
SEQ ID NO: 15
ISITEIKGVIVHRIETILF-eK-KKKIKHVPGGGSVQIVYK-NH2;
and
h.
SEQ ID NO: 16
ISITEIKGVIVHRIETILF-eK-KKKVSGDTSPRHLSNVSST-NH2

(18) A composition comprising a Tau peptide immunogen construct according to (17).

(19) A pharmaceutical composition comprising:
a. a Tau peptide immunogen construct according to (17); and
b. and a pharmaceutically acceptable delivery vehicle and/or adjuvant.

(20) The pharmaceutical composition of (19), wherein the Tau peptide immunogen construct is mixed with an CpG oligodeoxynucleotide (ODN), e.g., CpG1, CpG2, or CpG3, to form a stabilized immunostimulatory complex.

(21) The pharmaceutical composition of (19) or (20), wherein the composition comprises an aluminum-based adjuvant (aluminum phosphate or aluminum hydroxide) or another adjuvant described herein.

(22) A method of preventing, inhibiting, reducing the severity of, delaying, or treating a tauopathy in a subject, the method comprising administering a tau peptide immunogen construct or a composition of any one (1) to (21) to the subject.

(23) The method of (22), wherein the tauopathy is selected from the group consisting of Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

### Example 1

The accumulation of misfolded Tau in the brain correlates with clinical decline in Alzheimer's Disease (AD) but manifests decades earlier than cognitive symptoms (Congdon and Sigurdsson, Nat. Rev. Neurol, 2018). Targeting pathological Tau prior to the clinical onset of AD could help prevent disease and/or progression. We have developed a vaccine-based immunotherapy approach for the prevention and treatment of Alzheimer's Disease. The results of preclinical characterization of Tau vaccines are presented in Table A, below. The peptides noted in the Figures can be cross-referenced based on column 1 of Table 1.

TABLE A
Immunogenicity of Tau candidates

| Construct | UbTh1 | CpG1 | Antigen | Mono | PFF |
|---|---|---|---|---|---|
| A | 10 | 107 | 396 | 504 | 17,400 |
| B | 10 | 221 | 1,490 | 3,512 | 2,060 |
| C | 10 | 199 | 2,827 | 5,732 | 745 |
| D | 17 | 18 | 10,086 | 574 | 601 |
| E | 10 | 15 | 5,216 | 554 | 498 |
| F | 10 | 39 | 97 | 2,782 | 4,050 |
| G | 10 | 59 | 4,003 | 3,299 | 4,556 |

Ec50 values generated via 12-point dilution curve ELISAs probing against the T helper peptide, CpG, Tau construct-specific peptides, full length monomeric Tau (Mono) and Tau preformed fibrils (PFF) using the terminal bleed sera (15 weeks) from vaccinated male Hartley guinea pigs.

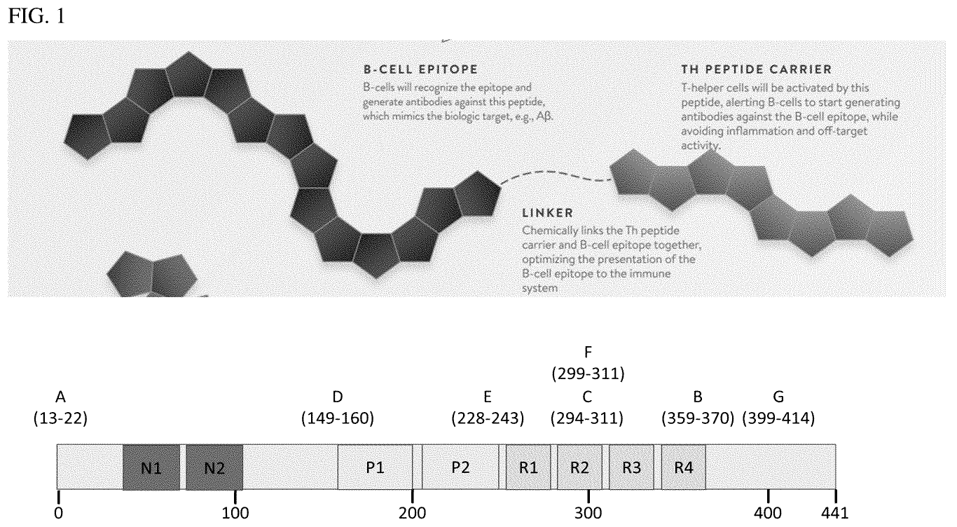

The platform upon which the vaccines is based is illustrated in FIG. 1, and includes a B-cell epitope, a spacer or linker, and a Th peptide carrier. B-cells will recognize the B-cell epitope and generate antibodies against the peptide, which mimics the biologic target (e.g., Tau). The spacer chemically links the B-cell epitope to the Th peptide, thereby optimizing the presentation of the B-cell epitope to the immune system. The Th peptide carrier activates T-helper cells, which alert B-cells to start generating antibodies against the B-cell epitope, while avoiding inflammation and off-target activity. The primary structure of Tau, with functional domains and targeting peptides annotated, is shown at the bottom of FIG. 1. The peptides indicated in this figure, as well as the other figures set forth herein, are as follows:

Constructs were formulated in Adju-Phos CpG1 at 100 μg/mL+300 μg/0.25 mL peptide. Guinea pigs were given 5 intramuscular shots 3 weeks apart, with the terminal bleed collected at 15 wpi. Antibodies against the T helper peptide, adjuvant components, and Tau were quantified by ELISA in serial dilution. In addition, antibody binding was characterized against recombinant and brain-derived Tau preps by Western blot, dot blot, and biolayer interferometry (BLI). Assessments of in vitro function were made via Tau FRET-aggregation and pHrodo uptake assay in HEK293 and B103 cells.

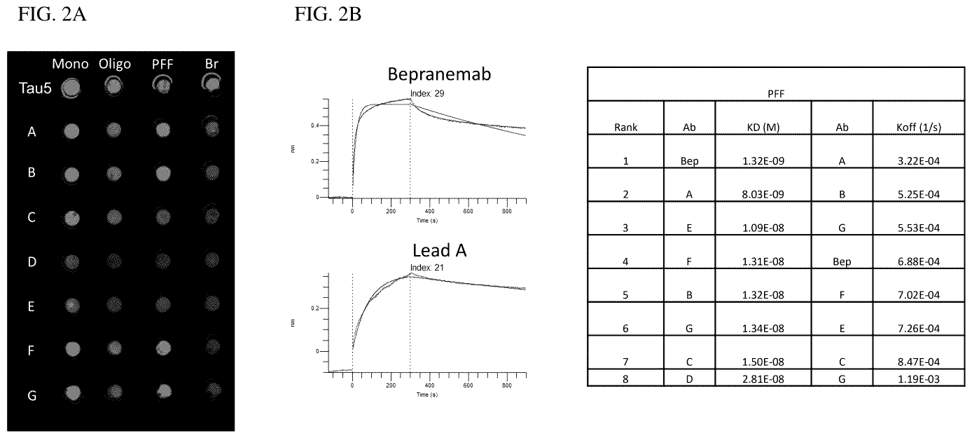

The results of antibody binding characterization are shown in FIGS. 2A and 2B. The binding potency of vaccine-derived antibodies against three forms of recombinant Tau (monomeric, oligomeric, and PFF) and a sarkosyl extract from postmortem brain tissue (Br) were characterized via dotblot (FIG. 2A). Diverse binding profiles were observed against the different forms of Tau. BLI results show antibody binding to Tau forms with from constructs performing with Kd in nM range, with three constructs having slower off rates than Bepranemab (FIG. 2B). Representative binding curves of Bep and construct A to PFF are presented.

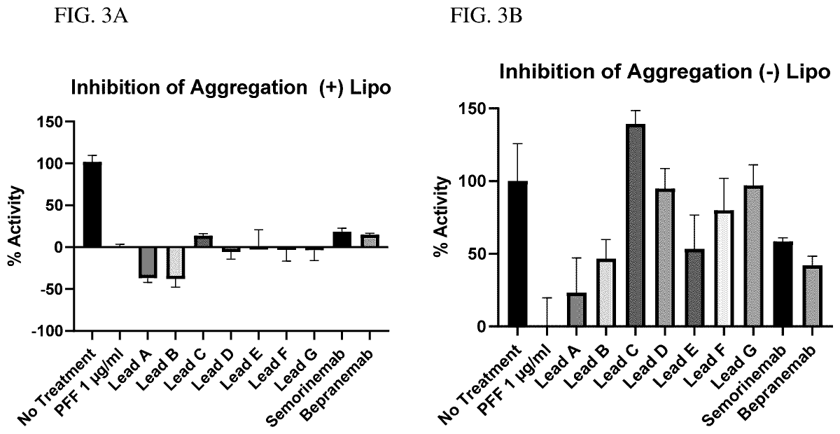

The results of an aggregation assay using a Tau biosensor line are shown in FIGS. 3A and 3B. Tau biosensor lines (Frost et al., J. Biol. Chem., 2009) were used to assess the functional inhibition of vaccine derived antibodies compared to anti-Tau mAbs, Semorinemab, and Bepranemab. Assay conditions included the addition of lipofectamine (FIG. 3A) and without lipofectamine (FIG. 3B). Only the condition without lipofectamine resulted in Abs inhibiting aggregation.

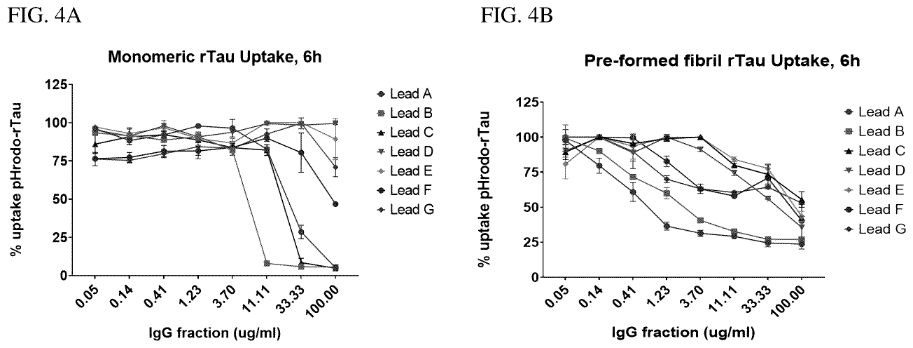

The results of a Tau uptake assay are shown in FIGS. 4A and 4B. B103 cells were exposed to pHrodo labeled Tau preparations for 6h. Upon uptake, pHrodo-Tau fluoresces, which enabled quantification using the IncuCyte live cell imager. Constructs A, B, C, and F inhibit the uptake of monomeric Tau in dose response (FIG. 4A). All constructs resulted in a dose-dependent reduction of PFF uptake (FIG. 4B). Constructs vary in potency; however, all exhibited stronger inhibition of PFF than monomeric Tau uptake.

The antibodies from the Tau candidates display diverse binding profiles against different forms of Tau. No significant immunogenicity was observed against the Th1 peptides or CpG1. Functional assays illustrate that binding antibodies prevent aggregation by inhibiting uptake.

### Example 2

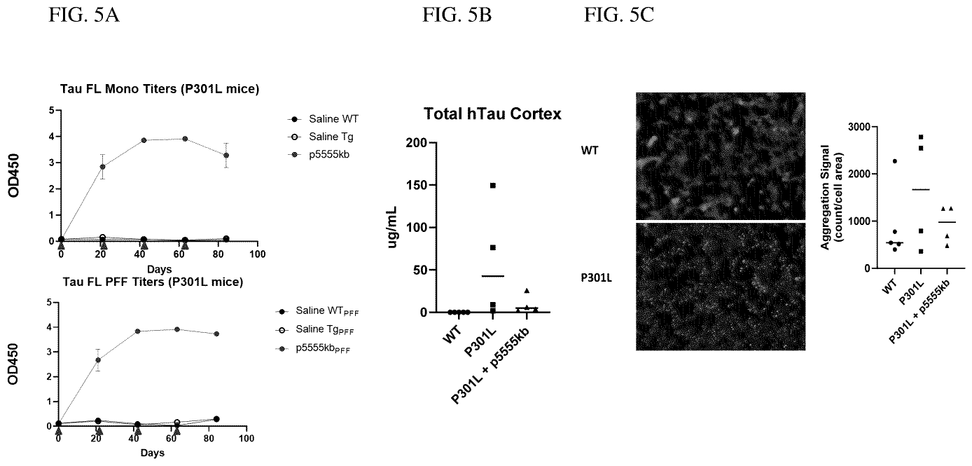

Vaccination studies in WT and P301L mice were carried out. FIG. 5A shows that P301L mice vaccinated with p5555 kb generate robust titers against monomeric and PFF Tau. FIG. 5B shows that vaccination with p5555 kb shows reduced Tau accumulation in P301L. FIG. 5C shows that lysates from brains of P301L mice vaccinated with p5555 kb exhibit evidence of reduced induction of aggregation in a Tau biosensor line. These results show that both WT and P301L mice demonstrate positive responses to vaccination with p5555 kb.

### Example 3

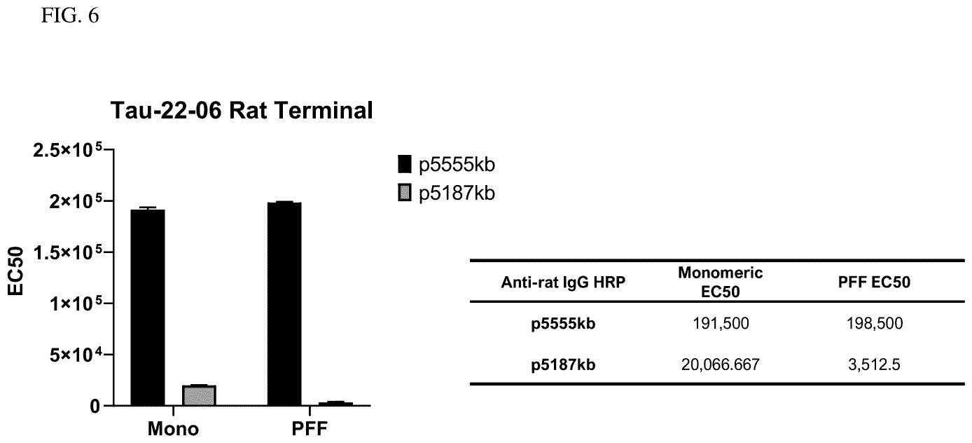

Immunogenicity of p5555 kb and p5187 kb was assessed in rats. The results are shown in FIG. 6. The ELISA analysis shows that titers were on par with guinea pig studies. These constructs therefore have immunogenicity in different species.

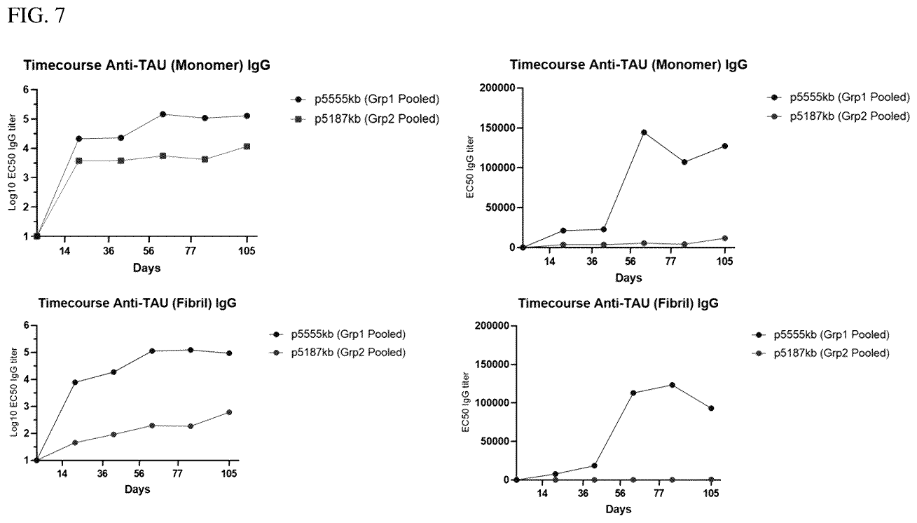

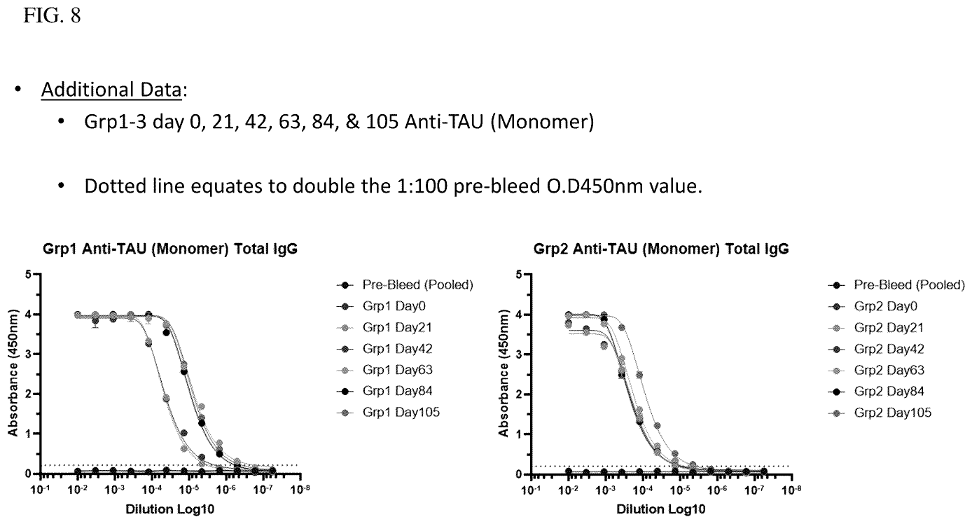

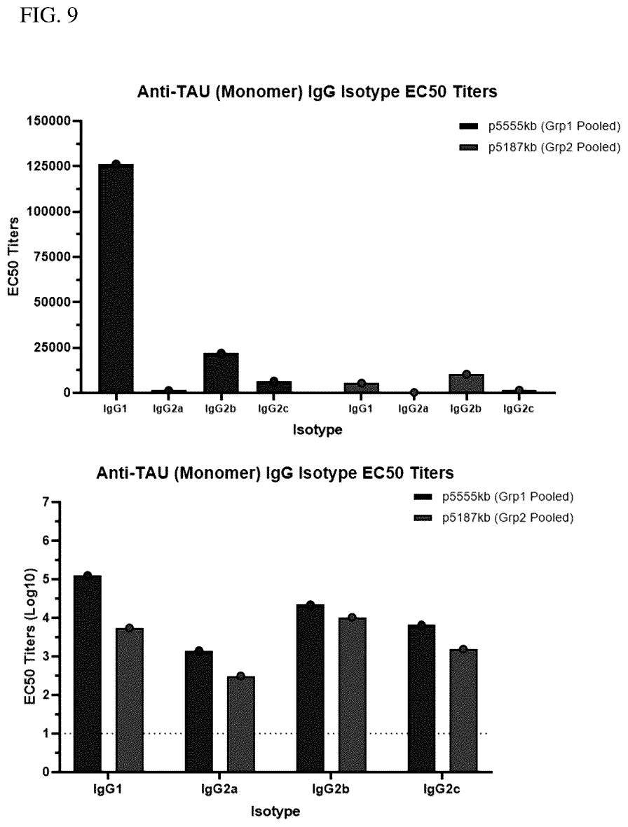

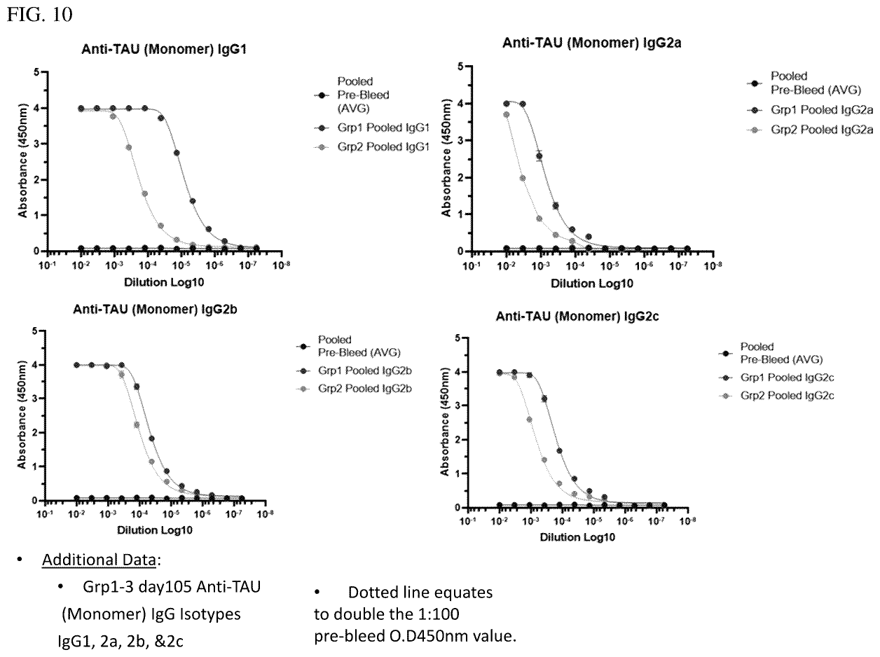

In further studies, isotype analysis of antibodies generated in rats was carried out. Ten male Sprague Dawley rates, 200-250 g, per group, 2 groups (20 rats) were used. Vaccines were dosed at weeks 0, 3, 6, 9, and 12 (intramuscular, 0.25 mL/dose). The bleed schedule was pre-dose on weeks 0, 3, 6, 9, and 12; terminal bleed on week 15. Blood samples were separated into serum and placed on dry ice prior to subsequent thawing and analysis. Group 1: p5555 kb, 10 animals, 30 μg/0.25 mL/dose/IM (Adjuphos, CpG1), Group 2: p5187, 10 animals, 30 μg/0.25 mL/dose/IM (Adjuphos, CpG1). The results are shown in FIGS. 7-10, and show (i) Group 1 (p5555 kb) had significantly higher total IgG titers at post day 42 time points, (ii) IgG1 is the predominant isotype in all groups, followed by IgG2b, IgG2c, and IgG2a, suggesting a TH-2 response, and (iii) antibody titers plateau (104-5) after the day 42 time point (2nd boost) for Groups 1 and 2.

In additional studies, western blot analysis was carried out to assess antibody binding to brain tissue (normal hippocampus, normal temporal lobe, diseased hippocampus, and diseased temporal lobe). The data is consistent with guinea pig data, as p5187 and p5555 rat IgG binds well to human brain lysates

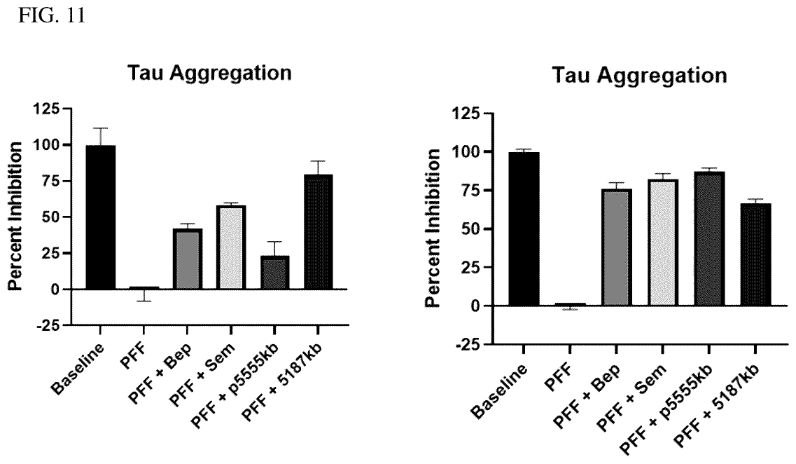

Tau aggregation assays show that rat TgG from p5555 and p5187 inhibit tau aggregation at levels comparable to the monoclonals SemorinemAb and BeprenemAb (FIG. 11).

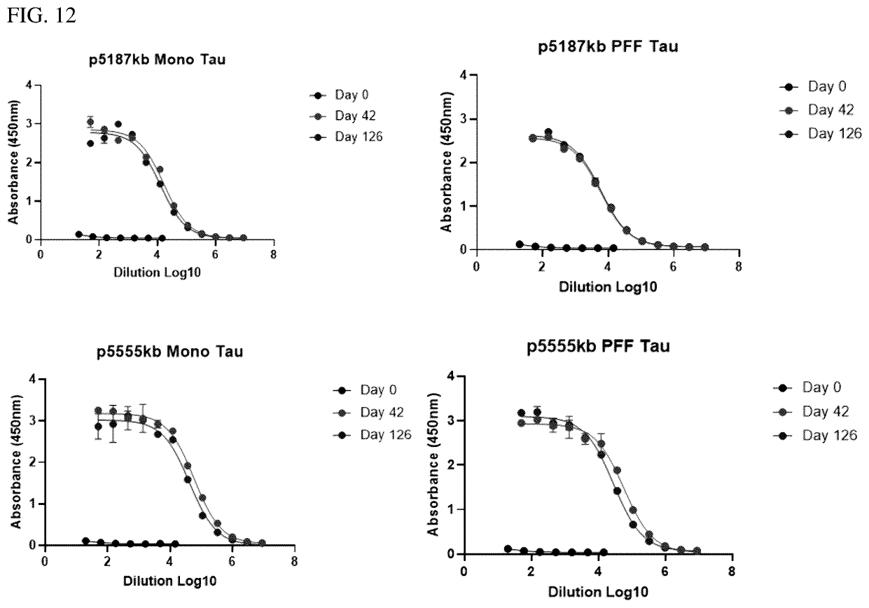

In further studies, immunogenicity of p5555 kb and p5187 kb was assessed in mice. The results are set forth in FIG. 12 and show that robust titers are obtained with p5555 and p5187 against PFF and monomeric forms of tau.

TABLE 1
Amino Acid Sequences of Immunogenic Constructs

| | | | Target |
|---|---|---|---|
| | | | sequence |
| | Designation | Sequences | region |
| | p5459kb | MAEPRQEFEVMEDHAGTYGLGDKKK- | 1-22 |
| | | eK-ISITEIKGVIVHRIETILF-NH2 | |
| A | p5555kb | DHAGTYGLGDKKK-eK- | 13-22 |
| | | ISITEIKGVIVHRIETILF-NH2 | |
| B | p5404kb | ISITEIKGVIVHRIETILF-eK- | 359-370 |
| | | KKKNITHVPGGGNKK-NH2 | |
| C | p5137kb | ISITEIKGVIVHRIETILF-eK- | 294-311 |
| | | KKKKDNIKHVPGGGSVQIVYK-NH2 | |
| D | p5505kb | ISITEIKGVIVHRIETILF-eK- | 149-160 |
| | | KKKTKIATPRGAAPP-NH2 | |
| E | p4598kb | ISITEIKGVIVHRIETILF-eK- | 228-243 |
| | | KKKVVRTPPKSPSSAKSRL-NH2 | |
| F | p5187kb | ISITEIKGVIVHRIETILF-eK- | 299-311 |
| | | KKKIKHVPGGGSVQIVYK-NH2 | |
| G | p5506kb | ISITEIKGVIVHRIETILF-eK- | 399-414 |
| | | KKKVSGDTSPRHLSNVSST-NH2 | |

The target peptide sequences are underlined and are SEQ ID NOs: 1-8, respectively (going from the top of the table to the bottom). The immunogenic construct sequences are SEQ ID NOs: 9-16, respectively (going from the top of the table to the bottom and referring to the column noted as “Sequences”)

TABLE 2
Amino Acid Sequences of Pathogen Protein Derived
Th Epitopes Including Idealized Artificial
Th Epitopes for Employment in the Design of Tau
Peptide Immunogen Constructs

| | | SEQ |
|---|---|---|
| | | ID |
| Description | Sequence | NO |
| Clostridium | KKQYIKANSKFIGITEL | 138 |
| tetani1 Th | | |
| MvF1 Th | LSEIKGVIVHRLEGV | 139 |
| Bordetella | GAYARCPNGTRALTVAELRGNAEL | 140 |
| pertussis Th | | |
| Clostridium | WVRDIIDDFTNESSQKT | 141 |
| tetani2 Th | | |
| Diphtheria Th | DSETADNLEKTVAALSILPGHGC | 142 |
| Plasmodium | DHEKKHAKMEKASSVFNVVNS | 143 |
| falciparum Th | | |
| Schistosoma | KWFKTNAPNGVDEKHRH | 144 |
| mansoni Th | | |
| Cholera Toxin Th | ALNIWDRFDVFCTLGATTGYLKGNS | 145 |
| MvF2 Th | ISEIKGVIVHKIEGI | 146 |
| KKKMvF3 Th | KKKISISEIKGVIVHKIEGILF | 147 |
| | T RT TR T | |
| HbsAg1 Th | KKKLFLLTKLLTLPQSLD | 148 |
| | RRRIKII RII I L IR | |
| | VRVV VV V I V | |
| | F FF FF F V F | |
| MvF4 Th (UBITh®3) | ISISEIKGVIVHKIETILF | 149 |
| | T RT TR | |
| HbsAg2 Th | KKKIITITRIITIPQSLD | 150 |
| | FFLL L ITTI | |
| MvF5 Th (UBITh®1) | ISITEIKGVIVHRIETILF | 151 |
| HbsAg3 Th (UBITh®2) | KKKIITITRIITIITTID | 152 |
| Influenza MP1_1 Th | FVFTLTVPSER | 153 |
| Influenza MP1_2 Th | SGPLKAEIAQRLEDV | 154 |
| Influenza NSP1 Th | DRLRRDQKS | 155 |
| EBV BHRF1 Th | AGLTLSLLVICSYLFISRG | 156 |
| Clostridium tetani | QYIKANSKFIGITEL | 157 |
| TT1 Th | | |
| EBV EBNA-1 Th | PGPLRESIVCYFMVFLQTHI | 158 |
| Clostridium tetani | FNNFTVSFWLRVPKVSASHLE | 159 |
| TT2 Th | | |
| Clostridium tetani | KFIIKRYTPNNEIDSF | 160 |
| TT3 Th | | |
| Clostridium tetani | VSIDKFRIFCKALNPK | 161 |
| TT4 Th | | |
| EBV CP Th | VPGLYSPCRAFFNKEELL | 162 |
| HCMV IE1 Th | DKREMWMACIKELH | 163 |
| EBV GP340 Th | TGHGARTSTEPTTDY | 164 |
| EBV BPLF1 Th | KELKRQYEKKLRQ | 165 |
| EBV EBNA-2 Th | TVFYNIPPMPL | 166 |

TABLE 3
Tau protein sequence, full length (441 aa)
isoform with 4R and 2N (GenBank: AGF19246.1)
Sequence (SEQ ID NO: 167)

| | |
|---|---|
| MAEPRQEFEVMEDHAGTYGLGDRKDQGGYTMHQDQEGDTDAGLKESPLQTPTEDGSEEPGSETSDAKSTP | 70 |
| TAEDVTAPLVDEGAPGKQAAAQPHTEIPEGTTAEEAGIGDTPSLEDEAAGHVTQARMVSKSKDGTGSDDK | 140 |
| KAKGADGKTKIATPRGAAPPGQKGQANATRIPAKTPPAPKTPPSSGEPPKSGDRSGYSSPGSPGTPGSRS | 210 |
| RTPSLPTPPTREPKKVAVVRTPPKSPSSAKSRLQTAPVPMPDLKNVKSKIGSTENLKHQPGGGKVQIINK | 280 |
| KLDLSNVQSKCGSKDNIKHVPGGGSVQIVYKPVDLSKVTSKCGSLGNIHHKPGGGQVEVKSEKLDFKDRV | 350 |
| QSKIGSLDNITHVPGGGNKKIETHKLTFRENAKAKTDHGAEIVYKSPVVSGDTSPRHLSNVSSTGSIDMV | 420 |
| DSPQLATLADEVSASLAKQGL | |

TABLE 4
Localization of immunogenic regions within Tau
275-311 through fine epitope mapping (SEQ ID NOs: 17-34)
Tau (275-311) B epitope mapping for p4604kb
Feb. 28, 2018

| | | | Hyperimmune serum |
|---|---|---|---|
| | | | PTAU-15-01GP grp |
| | | | 10 (4603 kb, UBITh1- |
| | | | ϵk-kkk-(V275-K311, |
| | 275 311 | | P301 → S301), 6w |
| | VQIINKKLDLSNVQSKCGSKDNIKHVPGGGSVQIVYK | | Abs (450 nm) |
| p5116kb: | KLDLSNVQSKCGSKDNIKHVPGGGSVQIVYK | (281-311) | 3.782 |
| p5117kb: | DLSNVQSKCGSKDNIKHVPGGGSVQIVYK | (283-311) | 3.802 |
| p5118kb: | SNVQSKCGSKDNIKHVPGGGSVQIVYK | (285-311) | 3.788 |
| p5119kb: | VQSKCGSKDNIKHVPGGGSVQIVYK | (287-311) | 3.836 |
| p5120kb: | SKCGSKDNIKHVPGGGSVQIVYK | (289-311) | 3.826 |
| p5121kb: | CGSKDNIKHVPGGGSVQIVYK | (291-311) | 3.781 |
| p5122kb: | SKDNIKHVPGGGSVQIVYK | (293-311) | 3.837 |
| p5123kb: | DNIKHVPGGGSVQIVYK | (295-311) | 3.732 |
| p5124kb: | IKHVPGGGSVQIVYK | (297-311) | 3.477 |
| p5125kb: | HVPGGGSVQIVYK | (299-311) | 0.221 |
| p5126kb: | PGGGSVQIVYK | (301-311) | 0.099 |
| p5127kb: | VQIINKKLDLSNVQSKCGSKDNIKHVPGGGS | (275-305) | 0.148 |
| p5128kb: | VQIINKKLDLSNVQSKCGSKDNIKHVP | (275-301) | 0.281 |
| p5129kb: | VQIINKKLDLSNVQSKCGSKDNI | (275-297) | 0.303 |
| p5130kb: | VQIINKKLDLSNVQSKCGS | (275-293) | 0.320 |
| p5131kb: | VQIINKKLDLSNVQS | (275-289) | 0.091 |
| p5132kb: | VQIINKKLDLS | (275-285) | 0.095 |

| | | |
|---|---|---|
| 4604a (V275-K311) | (275-311) | 3.994 |

Hyperimmune serums from:
Pooled group 10 (4603 kb, UBITh1-ϵk-kkk-(V275-K311, P301 → S301) of PTAU-15-01GP, 6 wpi.
Positive peptide: 4604a (V275-K311)
Hyperimmune serum 1/10000 dilution for mapping peptide
Hyperimmune serum 1/10000 dilution for positive peptide

1-23. (canceled)

24. A Tau peptide immunogen construct comprising:
a B cell epitope comprising SEQ ID NO: 2 or a functional analogue thereof; and
a heterologous T helper cell epitope selected from SEQ ID NOs: 138-166;
wherein the B cell epitope is covalently linked to the heterologous T helper cell epitope directly or through a heterologous spacer.

25. The Tau peptide immunogen construct of claim 24, which is represented by the formulae: 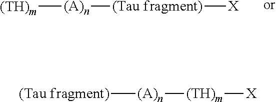
wherein
(Th) is the heterologous T helper cell epitope;
(A) is the heterologous spacer;
(Tau fragment) is the B cell epitope;
X is an α-COOH or α-CONH₂ of an amino acid;
m is from 1 to 4; and
n is from 0 to 10.

26. The Tau peptide immunogen construct of claim 24, wherein the B cell epitope is of the amino acid sequence of SEQ ID NO: 2.

27. The Tau peptide immunogen construct of claim 24, wherein the T helper cell epitope is selected from SEQ ID NOs: 149 and 151-152.

28. The Tau peptide immunogen construct of claim 24, wherein the T helper cell epitope is of the amino acid sequence of SEQ ID NO: 151.

29. The Tau peptide immunogen construct of claim 24, wherein the T helper cell epitope is of the amino acid sequence of SEQ ID NO: 151 and the B cell epitope is of the amino acid sequence of SEQ ID NO: 2.

30. The Tau peptide immunogen construct of claim 24, wherein the Tau peptide immunogen construct comprises the heterologous spacer, and the heterologous spacer is selected from an amino acid, Lys-, Gly-, Lys-Lys-Lys-, (α, ε-N) Lys, Lys-Lys-Lys-ε-N-Lys-, and ε-N-Lys-Lys-Lys-Lys-.

31. The Tau peptide immunogen construct of claim 24, wherein the Tau peptide immunogen construct comprises the heterologous spacer, and the heterologous spacer is Lys-Lys-Lys-ε-N-Lys-, or ε-N-Lys-Lys-Lys-Lys-.

32. The Tau peptide immunogen construct of claim 29, wherein the Tau peptide immunogen construct comprises the heterologous spacer, and the heterologous spacer is Lys-Lys-Lys-ε-N-Lys-, or ε-N-Lys-Lys-Lys-Lys-.

33. The Tau peptide immunogen construct of claim 24, wherein the Tau peptide immunogen construct comprises or is of the amino acid sequence of SEQ ID NO: 10, or comprises or is of the amino acid sequence of a construct in which the positions of the B cell epitope and the T helper cell epitope according to SEQ ID NO: 10 are reversed.

34. The Tau peptide immunogen construct of claim 24, wherein the Tau peptide immunogen construct is of the amino acid sequence of SEQ ID NO: 10.

35. A pharmaceutical composition comprising the Tau peptide immunogen construct of claim 24 and a pharmaceutically acceptable delivery vehicle and/or adjuvant.

36. The pharmaceutical composition of claim 35, wherein the Tau peptide immunogen construct is mixed with an CpG oligonucleotide (ODN) to form a stabilized immunostimulatory complex, optionally wherein the CpG ODN is CpG1, CpG2, or CpG3.

37. The pharmaceutical composition of claim 35, wherein the pharmaceutically acceptable delivery vehicle and/or adjuvant comprises an aluminum-based adjuvant, optionally wherein the aluminum-based adjuvant is aluminum phosphate or aluminum hydroxide.

38. A pharmaceutical composition comprising the Tau peptide immunogen construct of claim 33 and a pharmaceutically acceptable delivery vehicle and/or adjuvant.

39. The pharmaceutical composition of claim 38, wherein the Tau peptide immunogen construct is mixed with an CpG oligonucleotide (ODN) to form a stabilized immunostimulatory complex, optionally wherein the CpG ODN is CpG1, CpG2, or CpG3.

40. The pharmaceutical composition of claim 39, wherein the pharmaceutically acceptable delivery vehicle and/or adjuvant comprises an aluminum-based adjuvant, optionally wherein the aluminum-based adjuvant is aluminum phosphate or aluminum hydroxide.

41. An isolated antibody or epitope-binding fragment thereof elicited by the Tau peptide immunogen construct of claim 24.

42. A method of preventing, inhibiting, reducing the severity of, delaying, or treating a tauopathy in a subject, the method comprising administering the Tau peptide immunogen construct of claim 24 to the subject, optionally wherein the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

43. The method of claim 42, wherein the tauopathy is Alzheimer's disease.

44. A method of
(a) inhibiting Tau aggregation in a subject;
(b) inducing disaggregate of preformed Tau aggregates in a subject;
(c) reducing neurodegeneration triggered by exogenous Tau aggregates in a subject;
(d) reducing neurodegeneration in Tau overexpressing cells;
(e) reducing serum Tau levels in a subject;
(f) reducing oligomeric Tau level in the brain of a subject; or
(g) reducing neuropathology and recovery of motor activities in a subject;
the method comprising administering the Tau peptide immunogen construct of claim 24 to the subject.

45. A method of preventing, inhibiting, reducing the severity of, delaying, or treating a tauopathy in a subject, the method comprising administering the Tau peptide immunogen construct of claim 33 to the subject, optionally wherein the tauopathy is selected from Alzheimer's disease, Lewy body disease, frontotemporal dementia, parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, dementia pugilistica, Down syndrome, Gerstmann-Straussler-Scheinker disease, Hallerworden-Spatz disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

46. The method of claim 45, wherein the tauopathy is Alzheimer's disease.

47. A method of
(a) inhibiting Tau aggregation in a subject;
(b) inducing disaggregate of preformed Tau aggregates in a subject;
(c) reducing neurodegeneration triggered by exogenous Tau aggregates in a subject;
(d) reducing neurodegeneration in Tau overexpressing cells;
(e) reducing serum Tau levels in a subject;
(f) reducing oligomeric Tau level in the brain of a subject; or
(g) reducing neuropathology and recovery of motor activities in a subject;
the method comprising administering the Tau peptide immunogen construct of claim 33 to the subject.


SEQUENCE LISTING (172 sequences)

SEQ ID NO: 1 - protein, 22 aa, synthetic construct
MAEPRQEFEVMEDHAGTYGLGD

SEQ ID NO: 2 - protein, 10 aa, synthetic construct
DHAGTYGLGD

SEQ ID NO: 3 - protein, 12 aa, synthetic construct
NITHVPGGGNKK

SEQ ID NO: 4 - protein, 16 aa, synthetic construct
NIKHVPGGGSVQIVYK

SEQ ID NO: 5 - protein, 12 aa, synthetic construct
TKIATPRGAAPP

SEQ ID NO: 6 - protein, 16 aa, synthetic construct
VVRTPPKSPSSAKSRL

SEQ ID NO: 7 - protein, 15 aa, synthetic construct
IKHVPGGGSVQIVYK

SEQ ID NO: 8 - protein, 16 aa, synthetic construct
VSGDTSPRHLSNVSST

SEQ ID NO: 9 - protein, 45 aa, synthetic construct
  SITE 26: note = Epsilon lysine
  SITE 45: note = Amidated residue
MAEPRQEFEVMEDHAGTYGLGDKKKKISITEIKGVIVHRIETILF

SEQ ID NO: 10 - protein, 33 aa, synthetic construct
  SITE 14: note = Epsilon lysine
  SITE 33: note = Amidated residue
DHAGTYGLGDKKKKISITEIKGVIVHRIETILF

SEQ ID NO: 11 - protein, 35 aa, synthetic construct
  SITE 20: note = Epsilon lysine
  SITE 35: note = Amidated residue
ISITEIKGVIVHRIETILFKKKKNITHVPGGGNKK

SEQ ID NO: 12 - protein, 41 aa, synthetic construct
  SITE 20: note = Epsilon lysine
  SITE 41: note = Amidated residue
ISITEIKGVIVHRIETILFKKKKKDNIKHVPGGGSVQIVYK

SEQ ID NO: 13 - protein, 35 aa, synthetic construct
  SITE 20: note = Epsilon lysine
  SITE 35: note = Amidated residue
ISITEIKGVIVHRIETILFKKKKTKIATPRGAAPP

SEQ ID NO: 14 - protein, 39 aa, synthetic construct
  SITE 20: note = Epsilon lysine
  SITE 39: note = Amidated residue
ISITEIKGVIVHRIETILFKKKKVVRTPPKSPSSAKSRL

SEQ ID NO: 15 - protein, 38 aa, synthetic construct
  SITE 20: note = Epsilon lysine
  SITE 38: note = Amidated residue
ISITEIKGVIVHRIETILFKKKKIKHVPGGGSVQIVYK

SEQ ID NO: 16 - protein, 39 aa, synthetic construct
  SITE 20: note = Epsilon lysine
  SITE 39: note = Amidated residue
ISITEIKGVIVHRIETILFKKKKVSGDTSPRHLSNVSST

SEQ ID NO: 17 - protein, 37 aa, synthetic construct
VQIINKKLDLSNVQSKCGSKDNIKHVPGGGSVQIVYK

SEQ ID NO: 18 - protein, 31 aa, synthetic construct
KLDLSNVQSKCGSKDNIKHVPGGGSVQIVYK

SEQ ID NO: 19 - protein, 29 aa, synthetic construct
DLSNVQSKCGSKDNIKHVPGGGSVQIVYK

SEQ ID NO: 20 - protein, 27 aa, synthetic construct
SNVQSKCGSKDNIKHVPGGGSVQIVYK

SEQ ID NO: 21 - protein, 25 aa, synthetic construct
VQSKCGSKDNIKHVPGGGSVQIVYK

SEQ ID NO: 22 - protein, 23 aa, synthetic construct
SKCGSKDNIKHVPGGGSVQIVYK

SEQ ID NO: 23 - protein, 21 aa, synthetic construct
CGSKDNIKHVPGGGSVQIVYK

SEQ ID NO: 24 - protein, 19 aa, synthetic construct
SKDNIKHVPGGGSVQIVYK

SEQ ID NO: 25 - protein, 17 aa, synthetic construct
DNIKHVPGGGSVQIVYK

SEQ ID NO: 26 - protein, 15 aa, synthetic construct
IKHVPGGGSVQIVYK

SEQ ID NO: 27 - protein, 13 aa, synthetic construct
HVPGGGSVQIVYK

SEQ ID NO: 28 - protein, 11 aa, synthetic construct
PGGGSVQIVYK

SEQ ID NO: 29 - protein, 31 aa, synthetic construct
VQIINKKLDLSNVQSKCGSKDNIKHVPGGGS

SEQ ID NO: 30 - protein, 27 aa, synthetic construct
VQIINKKLDLSNVQSKCGSKDNIKHVP

SEQ ID NO: 31 - protein, 23 aa, synthetic construct
VQIINKKLDLSNVQSKCGSKDNI

SEQ ID NO: 32 - protein, 19 aa, synthetic construct
VQIINKKLDLSNVQSKCGS

SEQ ID NO: 33 - protein, 15 aa, synthetic construct
VQIINKKLDLSNVQS

SEQ ID NO: 34 - protein, 11 aa, synthetic construct
VQIINKKLDLS

SEQ ID NO: 35 - protein, 10 aa, synthetic construct
MAEPRQEFEV

SEQ ID NO: 36 - protein, 10 aa, synthetic construct
AEPRQEFEVM

SEQ ID NO: 37 - protein, 10 aa, synthetic construct
EPRQEFEVME

SEQ ID NO: 38 - protein, 10 aa, synthetic construct
PRQEFEVMED

SEQ ID NO: 39 - protein, 10 aa, synthetic construct
RQEFEVMEDH

SEQ ID NO: 40 - protein, 10 aa, synthetic construct
QEFEVMEDHA

SEQ ID NO: 41 - protein, 10 aa, synthetic construct
EFEVMEDHAG

SEQ ID NO: 42 - protein, 10 aa, synthetic construct
FEVMEDHAGT

SEQ ID NO: 43 - protein, 10 aa, synthetic construct
EVMEDHAGTY

SEQ ID NO: 44 - protein, 10 aa, synthetic construct
VMEDHAGTYG

SEQ ID NO: 45 - protein, 10 aa, synthetic construct
MEDHAGTYGL

SEQ ID NO: 46 - protein, 10 aa, synthetic construct
EDHAGTYGLG

SEQ ID NO: 47 - protein, 10 aa, synthetic construct
DHAGTYGLGD

SEQ ID NO: 48 - protein, 10 aa, synthetic construct
HAGTYGLGDR

SEQ ID NO: 49 - protein, 10 aa, synthetic construct
AGTYGLGDRK

SEQ ID NO: 50 - protein, 10 aa, synthetic construct
GTYGLGDRKD

SEQ ID NO: 51 - protein, 10 aa, synthetic construct
TYGLGDRKDQ

SEQ ID NO: 52 - protein, 10 aa, synthetic construct
YGLGDRKDQG

SEQ ID NO: 53 - protein, 10 aa, synthetic construct
GLGDRKDQGG

SEQ ID NO: 54 - protein, 10 aa, synthetic construct
LGDRKDQGGY

SEQ ID NO: 55 - protein, 10 aa, synthetic construct
GDRKDQGGYT

SEQ ID NO: 56 - protein, 10 aa, synthetic construct
DRKDQGGYTM

SEQ ID NO: 57 - protein, 10 aa, synthetic construct
RKDQGGYTMH

SEQ ID NO: 58 - protein, 10 aa, synthetic construct
KDQGGYTMHQ

SEQ ID NO: 59 - protein, 10 aa, synthetic construct
DQGGYTMHQD

SEQ ID NO: 60 - protein, 10 aa, synthetic construct
QGGYTMHQDQ

SEQ ID NO: 61 - protein, 10 aa, synthetic construct
GGYTMHQDQE

SEQ ID NO: 62 - protein, 10 aa, synthetic construct
GYTMHQDQEG

SEQ ID NO: 63 - protein, 10 aa, synthetic construct
YTMHQDQEGD

SEQ ID NO: 64 - protein, 10 aa, synthetic construct
TMHQDQEGDT

SEQ ID NO: 65 - protein, 10 aa, synthetic construct
MHQDQEGDTD

SEQ ID NO: 66 - protein, 10 aa, synthetic construct
HQDQEGDTDA

SEQ ID NO: 67 - protein, 10 aa, synthetic construct
QDQEGDTDAG

SEQ ID NO: 68 - protein, 10 aa, synthetic construct
DQEGDTDAGL

SEQ ID NO: 69 - protein, 10 aa, synthetic construct
QEGDTDAGLK

SEQ ID NO: 70 - protein, 10 aa, synthetic construct
EGDTDAGLKE

SEQ ID NO: 71 - protein, 45 aa, synthetic construct
MAEPRQEFEVMEDHAGTYGLGDRKDQGGYTMHQDQEGDTDAGLKE

SEQ ID NO: 72 - protein, 35 aa, synthetic construct
GSDDKKAKGADGKTKIATPRGAAPPGQKGQANATR

SEQ ID NO: 73 - protein, 10 aa, synthetic construct
GSDDKKAKGA

SEQ ID NO: 74 - protein, 10 aa, synthetic construct
SDDKKAKGAD

SEQ ID NO: 75 - protein, 10 aa, synthetic construct
DDKKAKGADG

SEQ ID NO: 76 - protein, 10 aa, synthetic construct
DKKAKGADGK

SEQ ID NO: 77 - protein, 10 aa, synthetic construct
KKAKGADGKT

SEQ ID NO: 78 - protein, 10 aa, synthetic construct
KAKGADGKTK

SEQ ID NO: 79 - protein, 10 aa, synthetic construct
AKGADGKTKI

SEQ ID NO: 80 - protein, 10 aa, synthetic construct
KGADGKTKIA

SEQ ID NO: 81 - protein, 10 aa, synthetic construct
GADGKTKIAT

SEQ ID NO: 82 - protein, 10 aa, synthetic construct
ADGKTKIATP

SEQ ID NO: 83 - protein, 10 aa, synthetic construct
DGKTKIATPR

SEQ ID NO: 84 - protein, 10 aa, synthetic construct
GKTKIATPRG

SEQ ID NO: 85 - protein, 10 aa, synthetic construct
KTKIATPRGA

SEQ ID NO: 86 - protein, 10 aa, synthetic construct
TKIATPRGAA

SEQ ID NO: 87 - protein, 10 aa, synthetic construct
KIATPRGAAP

SEQ ID NO: 88 - protein, 10 aa, synthetic construct
IATPRGAAPP

SEQ ID NO: 89 - protein, 10 aa, synthetic construct
ATPRGAAPPG

SEQ ID NO: 90 - protein, 10 aa, synthetic construct
TPRGAAPPGQ

SEQ ID NO: 91 - protein, 10 aa, synthetic construct
PRGAAPPGQK

SEQ ID NO: 92 - protein, 10 aa, synthetic construct
RGAAPPGQKG

SEQ ID NO: 93 - protein, 10 aa, synthetic construct
GAAPPGQKGQ

SEQ ID NO: 94 - protein, 10 aa, synthetic construct
AAPPGQKGQA

SEQ ID NO: 95 - protein, 10 aa, synthetic construct
APPGQKGQAN

SEQ ID NO: 96 - protein, 10 aa, synthetic construct
PPGQKGQANA

SEQ ID NO: 97 - protein, 10 aa, synthetic construct
PGQKGQANAT

SEQ ID NO: 98 - protein, 10 aa, synthetic construct
GQKGQANATR

SEQ ID NO: 99 - protein, 16 aa, synthetic construct
ADGKTKIATPRGAAPP

SEQ ID NO: 100 - protein, 45 aa, synthetic construct
IETHKLTFRENAKAKTDHGAEIVYKSPVVSGDTSPRHLSNVSSTG

SEQ ID NO: 101 - protein, 10 aa, synthetic construct
IETHKLTFRE

SEQ ID NO: 102 - protein, 10 aa, synthetic construct
ETHKLTFREN

SEQ ID NO: 103 - protein, 10 aa, synthetic construct
THKLTFRENA

SEQ ID NO: 104 - protein, 10 aa, synthetic construct
HKLTFRENAK

SEQ ID NO: 105 - protein, 10 aa, synthetic construct
KLTFRENAKA

SEQ ID NO: 106 - protein, 10 aa, synthetic construct
LTFRENAKAK

SEQ ID NO: 107 - protein, 10 aa, synthetic construct
TFRENAKAKT

SEQ ID NO: 108 - protein, 10 aa, synthetic construct
FRENAKAKTD

SEQ ID NO: 109 - protein, 10 aa, synthetic construct
RENAKAKTDH

SEQ ID NO: 110 - protein, 10 aa, synthetic construct
ENAKAKTDHG

SEQ ID NO: 111 - protein, 10 aa, synthetic construct
NAKAKTDHGA

SEQ ID NO: 112 - protein, 10 aa, synthetic construct
AKAKTDHGAE

SEQ ID NO: 113 - protein, 10 aa, synthetic construct
KAKTDHGAEI

SEQ ID NO: 114 - protein, 10 aa, synthetic construct
AKTDHGAEIV

SEQ ID NO: 115 - protein, 10 aa, synthetic construct
KTDHGAEIVY

SEQ ID NO: 116 - protein, 10 aa, synthetic construct
TDHGAEIVYK

SEQ ID NO: 117 - protein, 10 aa, synthetic construct
DHGAEIVYKS

SEQ ID NO: 118 - protein, 10 aa, synthetic construct
HGAEIVYKSP

SEQ ID NO: 119 - protein, 10 aa, synthetic construct
GAEIVYKSPV

SEQ ID NO: 120 - protein, 10 aa, synthetic construct
AEIVYKSPVV

SEQ ID NO: 121 - protein, 10 aa, synthetic construct
EIVYKSPVVS

SEQ ID NO: 122 - protein, 10 aa, synthetic construct
IVYKSPVVSG

SEQ ID NO: 123 - protein, 10 aa, synthetic construct
VYKSPVVSGD

SEQ ID NO: 124 - protein, 10 aa, synthetic construct
YKSPVVSGDT

SEQ ID NO: 125 - protein, 10 aa, synthetic construct
KSPVVSGDTS

SEQ ID NO: 126 - protein, 10 aa, synthetic construct
SPVVSGDTSP

SEQ ID NO: 127 - protein, 10 aa, synthetic construct
PVVSGDTSPR

SEQ ID NO: 128 - protein, 10 aa, synthetic construct
VVSGDTSPRH

SEQ ID NO: 129 - protein, 10 aa, synthetic construct
VSGDTSPRHL

SEQ ID NO: 130 - protein, 10 aa, synthetic construct
SGDTSPRHLS

SEQ ID NO: 131 - protein, 10 aa, synthetic construct
GDTSPRHLSN

SEQ ID NO: 132 - protein, 10 aa, synthetic construct
DTSPRHLSNV

SEQ ID NO: 133 - protein, 10 aa, synthetic construct
TSPRHLSNVS

SEQ ID NO: 134 - protein, 10 aa, synthetic construct
SPRHLSNVSS

SEQ ID NO: 135 - protein, 10 aa, synthetic construct
PRHLSNVSST

SEQ ID NO: 136 - protein, 10 aa, synthetic construct
RHLSNVSSTG

SEQ ID NO: 137 - protein, 30 aa, synthetic construct
RENAKAKTDHGAEIVYKSPVVSGDTSPRHL

SEQ ID NO: 138 - protein, 17 aa, synthetic construct
KKQYIKANSKFIGITEL

SEQ ID NO: 139 - protein, 15 aa, synthetic construct
LSEIKGVIVHRLEGV

SEQ ID NO: 140 - protein, 24 aa, synthetic construct
GAYARCPNGTRALTVAELRGNAEL

SEQ ID NO: 141 - protein, 17 aa, synthetic construct
WVRDIIDDFTNESSQKT

SEQ ID NO: 142 - protein, 23 aa, synthetic construct
DSETADNLEKTVAALSILPGHGC

SEQ ID NO: 143 - protein, 21 aa, synthetic construct
DHEKKHAKMEKASSVFNVVNS

SEQ ID NO: 144 - protein, 17 aa, synthetic construct
KWFKTNAPNGVDEKHRH

SEQ ID NO: 145 - protein, 25 aa, synthetic construct
ALNIWDRFDVFCTLGATTGYLKGNS

SEQ ID NO: 146 - protein, 15 aa, synthetic construct
ISEIKGVIVHKIEGI

SEQ ID NO: 147 - protein, 22 aa, synthetic construct
  VARIANT 7: note = S or T
  VARIANT 10: note = K or R
  VARIANT 11: note = G or T
  VARIANT 15: note = H or T
  VARIANT 16: note = K or R
  VARIANT 19: note = G or T
KKKISIXEIXXVIVXXIEXILF

SEQ ID NO: 148 - protein, 18 aa, synthetic construct
  VARIANT 1: note = K or R
  VARIANT 2: note = K or R
  VARIANT 3: note = K or R
  VARIANT 4: note = L, I, V, or F
  VARIANT 5: note = F, K, or R
  VARIANT 6: note = L, I, V, or F
  VARIANT 7: note = L, I, V, or F
  VARIANT 9: note = K or R
  VARIANT 10: note = L, I, V, or F
  VARIANT 11: note = L, I, V, or F
  VARIANT 13: note = L, I, V, or F
  VARIANT 15: note = Q, L, I, V, or F
  VARIANT 17: note = L, I, V, or F
  VARIANT 18: note = D or R
XXXXXXXTXXXTXPXSXX

SEQ ID NO: 149 - protein, 19 aa, synthetic construct
  VARIANT 4: note = S or T
  VARIANT 7: note = K or R
  VARIANT 8: note = G or T
  VARIANT 12: note = H or T
  VARIANT 13: note = K or R
ISIXEIXXVIVXXIETILF

SEQ ID NO: 150 - protein, 18 aa, synthetic construct
  VARIANT 4: note = I or F
  VARIANT 5: note = I or F
  VARIANT 6: note = T or L
  VARIANT 7: note = I or L
  VARIANT 11: note = I or L
  VARIANT 14: note = P or I
  VARIANT 15: note = Q or T
  VARIANT 16: note = S or T
  VARIANT 17: note = L or I
KKKXXXXTRIXTIXXXXD

SEQ ID NO: 151 - protein, 19 aa, synthetic construct
ISITEIKGVIVHRIETILF

SEQ ID NO: 152 - protein, 18 aa, synthetic construct
KKKIITITRIITIITTID

SEQ ID NO: 153 - protein, 11 aa, synthetic construct
FVFTLTVPSER

SEQ ID NO: 154 - protein, 15 aa, synthetic construct
SGPLKAEIAQRLEDV

SEQ ID NO: 155 - protein, 9 aa, synthetic construct
DRLRRDQKS

SEQ ID NO: 156 - protein, 19 aa, synthetic construct
AGLTLSLLVICSYLFISRG

SEQ ID NO: 157 - protein, 15 aa, synthetic construct
QYIKANSKFIGITEL

SEQ ID NO: 158 - protein, 20 aa, synthetic construct
PGPLRESIVCYFMVFLQTHI

SEQ ID NO: 159 - protein, 21 aa, synthetic construct
FNNFTVSFWLRVPKVSASHLE

SEQ ID NO: 160 - protein, 16 aa, synthetic construct
KFIIKRYTPNNEIDSF

SEQ ID NO: 161 - protein, 16 aa, synthetic construct
VSIDKFRIFCKALNPK

SEQ ID NO: 162 - protein, 18 aa, synthetic construct
VPGLYSPCRAFFNKEELL

SEQ ID NO: 163 - protein, 14 aa, synthetic construct
DKREMWMACIKELH

SEQ ID NO: 164 - protein, 15 aa, synthetic construct
TGHGARTSTEPTTDY

SEQ ID NO: 165 - protein, 13 aa, synthetic construct
KELKRQYEKKLRQ

SEQ ID NO: 166 - protein, 11 aa, synthetic construct
TVFYNIPPMPL

SEQ ID NO: 167 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 168 - protein, 6 aa, synthetic construct
  VARIANT 3: note = Any amino acid
  VARIANT 5: note = Any amino acid
PPXPXP

SEQ ID NO: 169 - protein, 4 aa, synthetic construct
  SITE 1: note = Epsilon lysine
KKKK

SEQ ID NO: 170 - DNA, 32 bp, synthetic construct
  source 1..32: mol_type = other DNA
tcgtcgttttgtcgttttgtcgttttgtcgtt

SEQ ID NO: 171 - DNA, 24 bp, synthetic construct
  source 1..24: mol_type = other DNA
tcgtcgttttgtcgttttgtcgtt

SEQ ID NO: 172 - DNA, 24 bp, synthetic construct
  source 1..24: mol_type = other DNA
tcgtcgttttgtcgttttgtcgtt